(19) United States
(12) Patent Application Publication
(10) Pub. No.: US 2017/0369860 A1
(43) Pub. Date: Dec. 28, 2017

(54) Endoglucanase Variants and Polynucleotides Encoding Same

(71) Applicant: Novozymes A/S, Bagsvaerd (DK)

(72) Inventors: Robert Blazej, San Francisco, CA (US); Charles Emrich, San Francisco, CA (US); Nicholas Toriello, San Francisco, CA (US); Hanshu Ding, Davis, CA (US); David Osborn, Sacramento, CA (US); Aubrey Jones, Davis, CA (US)

(73) Assignee: Novozymes A/S, Bagsvaerd (DK)

(21) Appl. No.: 15/538,816

(22) PCT Filed: Jan. 6, 2016
(86) PCT No.: PCT/US2016/012265

§ 371 Date: Jun. 22, 2017

Related U.S. Application Data
(60) Provisional application No. 62/095,609, filed on Dec. 22, 2014.

(51) Int. Cl. C12N 9/42; C12P 19/14; C12P 5/02; C12P 5/00; D21C 5/00; C12P 7/40; C12P 3/00

(52) U.S. Cl. CPC C12N 9/2437; D21C 5/005; C12Y 302/01004; C12P 19/14; C12P 7/40; C12P 5/023; C12P 5/007; C12P 3/00

(57) ABSTRACT

The present invention relates to endoglucanase variants. The present invention also relates to polynucleotides encoding the variants; nucleic acid constructs, expression vectors, and recombinant host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to endoglucanase variants, polynucleotides encoding the variants, methods of obtaining and producing the variants, and methods of using the variants.

### Description of the Related Art

Cellulose is a polymer of the simple sugar glucose covalently linked by beta-1,4-bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the lignocellulose is converted to fermentable sugars, e.g., glucose, the fermentable sugars can easily be fermented by yeast into ethanol.

WO 2012/088159 discloses variants of a Myceliophthora thermophila GH5 endoglucanase II.

There is a need in the art for endoglucanase variants with improved properties to increase the efficiency of saccharification of lignocellulosic feedstocks.

The present invention provides endoglucanase variants with improved properties.

### SUMMARY OF THE INVENTION

The present invention relates to isolated endoglucanase variants, comprising a substitution at one or more (e.g., several) positions corresponding to positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2, wherein the variants have endoglucanase activity.

The present invention also relates to isolated polynucleotides encoding the variants; nucleic acid constructs, expression vectors, and recombinant host cells comprising the polynucleotides; and methods of producing the variants.

The present invention also relates to processes for degrading a cellulosic material, comprising: treating the cellulosic material with an enzyme composition comprising an endoglucanase variant of the present invention. In one aspect, the processes further comprise recovering the degraded cellulosic material.

The present invention also relates to processes of producing a fermentation product, comprising: (a) saccharifying a cellulosic material with an enzyme composition comprising an endoglucanase variant of the present invention; (b) fermenting the saccharified cellulosic material with one or more (e.g., several) fermenting microorganisms to produce the fermentation product; and (c) recovering the fermentation product from the fermentation.

The present invention also relates to processes of fermenting a cellulosic material, comprising: fermenting the cellulosic material with one or more (e.g., several) fermenting microorganisms, wherein the cellulosic material is saccharified with an enzyme composition comprising an endoglucanase variant of the present invention. In one aspect, the fermenting of the cellulosic material produces a fermentation product. In another aspect, the processes further comprise recovering the fermentation product from the fermentation.

### Definitions

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc.).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C. and a suitable pH, such as 4-9, e.g., 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can be used in the presence of a soluble activating divalent metal cation according to WO 2008/151043 or WO 2012/122518, e.g., manganese or copper.

The AA9 polypeptide can also be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

Carbohydrate binding module: The term “carbohydrate binding module” means a domain within a carbohydrate-active enzyme that provides carbohydrate-binding activity (Boraston et al., 2004, Biochem. J. 383: 769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. Some CBMs are known to have specificity for cellulose.

Catalase: The term “catalase” means a hydrogen-peroxide:hydrogen-peroxide oxidoreductase (E.C. 1.11.1.6 or E.C. 1.11.1.21) that catalyzes the conversion of two hydrogen peroxides to oxygen and two waters.

Catalase activity can be determined by monitoring the degradation of hydrogen peroxide at 240 nm based on the following reaction:

2H₂O₂→2H₂O+O₂

The reaction is conducted in 50 mM phosphate pH 7 at 25° C. with 10.3 mM substrate (H₂O₂). Absorbance is monitored spectrophotometrically within 16-24 seconds, which should correspond to an absorbance reduction from 0.45 to 0.4. One catalase activity unit can be expressed as one μmole of H₂O₂ degraded per minute at pH 7.0 and 25° C.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No. 1 filter paper, microcrystalline cellulose, bacterial cellulose, algal cellulose, cotton, pretreated lignocellulose, etc. The most common total cellulolytic activity assay is the filter paper assay using Whatman No. 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc.).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, sugar cane straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Dissolved Oxygen Saturation Level: The saturation level of oxygen is determined at the standard partial pressure (0.21 atmosphere) of oxygen. The saturation level at the standard partial pressure of oxygen is dependent on temperature and solute concentrations. In an embodiment where the temperature during hydrolysis is 50° C., the saturation level would typically be in the range of 5-5.5 mg oxygen per kg slurry, depending on the solute concentrations. Hence, a concentration of dissolved oxygen of 0.5 to 10% of the saturation level at 50° C. corresponds to an amount of dissolved oxygen in a range from 0.025 ppm (0.5×5/100) to 0.55 ppm (10×5.5/100), such as, e.g., 0.05 to 0.165 ppm, and a concentration of dissolved oxygen of 10-70% of the saturation level at 50° C. corresponds to an amount of dissolved oxygen in a range from 0.50 ppm (10×5/100) to 3.85 ppm (70×5.5/100), such as, e.g., 1 to 2 ppm. In an embodiment, oxygen is added in an amount in the range of 0.5 to 5 ppm, such as 0.5 to 4.5 ppm, 0.5 to 4 ppm, 0.5 to 3.5 ppm, 0.5 to 3 ppm, 0.5 to 2.5 ppm, or 0.5 to 2 ppm.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can be determined using microcrystalline cellulose (AVICEL®) as substrate at pH 5 and 55° C. with shaking at 1000 rpm for 72 hours with enzyme loadings of 0.25-30 mg/g substrate as described in Example 5. Reducing sugars are quantitated using p-hydroxybenzoic acid hydrazide (PHBAH) according to Lever, 1072, Anal. Biochem. 47: 273-279.

Expression: The term “expression” includes any step involved in the production of a variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to control sequences that provide for its expression.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a polypeptide having one or more (e.g., several) amino acids absent from the amino and/or carboxyl terminus of the mature polypeptide thereof, wherein the fragment has endoglucanase activity. In one aspect, a fragment contains at least 85%, at least 90%, or at least 95% of the amino acid residues of the mature polypeptide.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 40° C., 45° C., 50° C., 55° C., 60° C., 65° C., 70° C., 75° C., or 80° C., and a suitable pH such as 4-9, e.g., 4.0, 4.5, 5.0, 5.5, 6.0, 6.5, 7.0, 7.5, 8.0, 8.5, or 9.0.

Hemicellulosic material: The term “hemicellulosic material” means any material comprising hemicelluloses. Hemicelluloses include xylan, glucuronoxylan, arabinoxylan, glucomannan, and xyloglucan. These polysaccharides contain many different sugar monomers. Sugar monomers in hemicellulose can include xylose, mannose, galactose, rhamnose, and arabinose. Hemicelluloses contain most of the D-pentose sugars. Xylose is in most cases the sugar monomer present in the largest amount, although in softwoods mannose can be the most abundant sugar. Xylan contains a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such an improved property is preferably increased specific performance.

Increased specific performance: The term “increased specific performance” by a variant of the present invention means improved conversion of a cellulosic material to a product, as compared to the same level of conversion by the parent. Increased specific performance is determined per unit protein (e.g., mg protein, or μmole protein). The increased specific performance of the variant relative to the parent can be assessed, for example, under one or more (e.g., several) conditions of pH, temperature, and substrate concentration. In one aspect, the product is glucose. In another aspect, the product is cellobiose. In another aspect, the product is glucose+cellobiose.

In one aspect, the condition is pH. For example, the pH can be any pH in the range of 3 to 7, e.g., 3.0, 3.5, 4.0, 4.5, 5.0, 5.5, 6.0, 6.5, or 7.0 (or in between). Any suitable buffer for achieving the desired pH can be used.

In another aspect, the condition is temperature. For example, the temperature can be any temperature in the range of 25° C. to 90° C., e.g., 25, 30, 35, 40, 45, 50, 55, 60, 65, 70, 75, 80, 85, or 90° C. (or in between).

In another aspect, the condition is substrate concentration. Any cellulosic material defined herein can be used as the substrate. In one aspect, the substrate concentration is measured as the dry solids content. The dry solids content is preferably in the range of about 1 to about 50 wt %, e.g., about 5 to about 45 wt %, about 10 to about 40 wt %, or about 20 to about 30 wt %. In another aspect, the substrate concentration is measured as the insoluble glucan content. The insoluble glucan content is preferably in the range of about 2.5 to about 25 wt %, e.g., about 5 to about 20 wt % or about 10 to about 15 wt %.

In another aspect, a combination of two or more (e.g., several) of the above conditions are used to determine the increased specific performance of the variant relative to the parent, such as any temperature in the range of 25° C. to 90° C., e.g., 25, 30, 35, 40, 45, 50, 55, 60, 65, 70, 75, 80, 85, or 90° C. (or in between) at a pH in the range of 3 to 7, e.g., 3.0, 3.5, 4.0, 4.5, 5.0, 5.5, 6.0, 6.5, or 7.0 (or in between).

The increased specific performance of the variant relative to the parent can be determined using any enzyme assay known in the art for endoglucanases as described herein. Alternatively, the increased specific performance of the variant relative to the parent can be determined using the assay described in Example 5 or 10.

In another aspect, the specific performance of the variant is at least 1.01-fold, e.g., at least 1.02-fold, at least 1.03-fold, at least 1.04-fold, at least 1.05-fold, at least 1.06-fold, at least 1.07-fold, at least 1.08-fold, at least 1.09-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 5-fold, at least 10-fold, at least 15-fold, at least 20-fold, at least 25-fold, and at least 50-fold higher than the specific performance of the parent.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Laccase: The term “laccase” means a benzenediol:oxygen oxidoreductase (E.C. 1.10.3.2) that catalyzes the following reaction: 1,2- or 1,4-benzenediol+O₂=1,2- or 1,4-benzosemiquinone+2 H₂O.

Laccase activity can be determined by measuring the oxidation of syringaldazine (4,4′-[azinobis(methanylylidene)]bis(2,6-dimethoxyphenol)) to the corresponding quinone 4,4′-[azobis(methanylylidene])bis(2,6-dimethoxycyclohexa-2,5-dien-1-one). The reaction (shown below) is detected by an increase in absorbance at 530 nm.

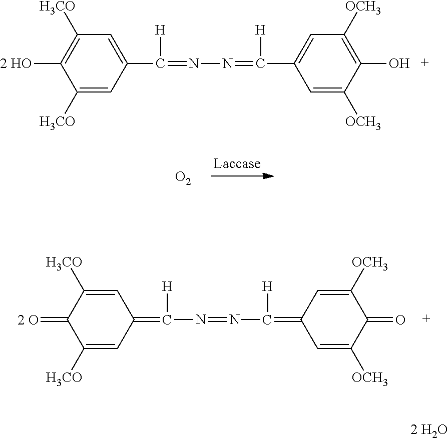

The reaction is conducted in 23 mM MES pH 5.5 at 30° C. with 19 μM substrate (syringaldazine) and 1 g/L polyethylene glycol (PEG) 6000. The sample is placed in a spectrophotometer and the change in absorbance is measured at 530 nm every 15 seconds up to 90 seconds. One laccase unit is the amount of enzyme that catalyzes the conversion of 1 μmole syringaldazine per minute under the specified analytical conditions.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 31 to 347 of SEQ ID NO: 2 based on the SignalP 3.0 program (Lo Leggio et al., 1997, Acta Crystallogr D Biol Crystallogr. 53: 599-605; Bendtsen et al., 2004, J. Mol. Biol. 340: 783-795) that predicts amino acids 1 to 30 of SEQ ID NO: 2 are a signal peptide. In another aspect, the mature polypeptide is amino acids 19 to 334 of SEQ ID NO: 4 based on the SignalP 3.0 program that predicts amino acids 1 to 18 of SEQ ID NO: 4 are a signal peptide. In another aspect, the mature polypeptide is amino acids 18 to 419 of SEQ ID NO: 6 based on the SignalP 3.0 program that predicts amino acids 1 to 17 of SEQ ID NO: 6 are a signal peptide. In another aspect, the mature polypeptide is amino acids 21 to 396 of SEQ ID NO: 8 based on the SignalP 3.0 program that predicts amino acids 1 to 20 of SEQ ID NO: 8 are a signal peptide. In another aspect, the mature polypeptide is amino acids 19 to 409 of SEQ ID NO: 10 based on the SignalP 3.0 program that predicts amino acids 1 to 18 of SEQ ID NO: 10 are a signal peptide. In another aspect, the mature polypeptide is amino acids 19 to 333 of SEQ ID NO: 12 based on the SignalP 3.0 program that predicts amino acids 1 to 18 of SEQ ID NO: 12 are a signal peptide. In another aspect, the mature polypeptide is amino acids 22 to 418 of SEQ ID NO: 14 based on the SignalP 3.0 program that predicts amino acids 1 to 21 of SEQ ID NO: 14 are a signal peptide. In another aspect, the mature polypeptide is amino acids 19 to 335 of SEQ ID NO: 16 based on the SignalP 3.0 program that predicts amino acids 1 to 18 of SEQ ID NO: 16 are a signal peptide. In another aspect, the mature polypeptide is amino acids 19 to 332 of SEQ ID NO: 18 based on the SignalP 3.0 program that predicts amino acids 1 to 18 of SEQ ID NO: 18 are a signal peptide. In another aspect, the mature polypeptide is amino acids 17 to 397 of SEQ ID NO: 20 based on the SignalP 3.0 program that predicts amino acids 1 to 16 of SEQ ID NO: 20 are a signal peptide. In another aspect, the mature polypeptide is amino acids 17 to 412 of SEQ ID NO: 22 based on the SignalP 3.0 program that predicts amino acids 1 to 16 of SEQ ID NO: 22 are a signal peptide. In another aspect, the mature polypeptide is amino acids 19 to 332 of SEQ ID NO: 24 based on the SignalP 3.0 program that predicts amino acids 1 to 18 of SEQ ID NO: 24 are a signal peptide. In another aspect, the mature polypeptide is amino acids 17 to 394 of SEQ ID NO: 26 based on the SignalP 3.0 program that predicts amino acids 1 to 16 of SEQ ID NO: 26 are a signal peptide. In another aspect, the mature polypeptide is amino acids 19 to 329 of SEQ ID NO: 28 based on the SignalP 3.0 program that predicts amino acids 1 to 18 of SEQ ID NO: 28 are a signal peptide. In another aspect, the mature polypeptide is amino acids 19 to 405 of SEQ ID NO: 30 based on the SignalP 3.0 program that predicts amino acids 1 to 18 of SEQ ID NO: 30 are a signal peptide. In another aspect, the mature polypeptide is amino acids 20 to 410 of SEQ ID NO: 32 based on the SignalP 3.0 program that predicts amino acids 1 to 19 of SEQ ID NO: 32 are a signal peptide. In another aspect, the mature polypeptide is amino acids 19 to 332 of SEQ ID NO: 34 based on the SignalP 3.0 program that predicts amino acids 1 to 18 of SEQ ID NO: 34 are a signal peptide. In another aspect, the mature polypeptide is amino acids 18 to 333 of SEQ ID NO: 36 based on the SignalP 3.0 program that predicts amino acids 1 to 17 of SEQ ID NO: 36 are a signal peptide. In another aspect, the mature polypeptide is amino acids 20 to 326 of SEQ ID NO: 38 based on the SignalP 3.0 program that predicts amino acids 1 to 19 of SEQ ID NO: 38 are a signal peptide. In another aspect, the mature polypeptide is amino acids 17 to 389 of SEQ ID NO: 40 based on the SignalP 3.0 program that predicts amino acids 1 to 16 of SEQ ID NO: 40 are a signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having endoglucanase activity. In one aspect, the mature polypeptide coding sequence is nucleotides 94 to 1041 of SEQ ID NO: 1 based on the SignalP 3.0 program (Bendtsen et al., 2004, supra) that predicts nucleotides 1 to 93 of SEQ ID NO: 1 encode a signal peptide. In another aspect, the mature polypeptide coding sequence is nucleotides 55 to 1002 of SEQ ID NO: 3 based on the SignalP 3.0 program that predicts nucleotides 1 to 54 of SEQ ID NO: 3 encode a signal peptide. In another aspect, the mature polypeptide coding sequence is nucleotides 52 to 1257 of SEQ ID NO: 5 based on the SignalP 3.0 program that predicts nucleotides 1 to 51 of SEQ ID NO: 5 encode a signal peptide. In another aspect, the mature polypeptide coding sequence is nucleotides 61 to 1188 of SEQ ID NO: 7 based on the SignalP 3.0 program that predicts nucleotides 1 to 60 of SEQ ID NO: 7 encode a signal peptide. In another aspect, the mature polypeptide coding sequence is nucleotides 55 to 1227 of SEQ ID NO: 9 based on the SignalP 3.0 program that predicts nucleotides 1 to 54 of SEQ ID NO: 9 encode a signal peptide. In another aspect, the mature polypeptide coding sequence is nucleotides 55 to 999 of SEQ ID NO: 11 based on the SignalP 3.0 program that predicts nucleotides 1 to 54 of SEQ ID NO: 11 encode a signal peptide. In another aspect, the mature polypeptide coding sequence is nucleotides 64 to 1254 of SEQ ID NO: 13 based on the SignalP 3.0 program that predicts nucleotides 1 to 63 of SEQ ID NO: 13 encode a signal peptide. In another aspect, the mature polypeptide coding sequence is nucleotides 55 to 1005 of SEQ ID NO: 15 based on the SignalP 3.0 program that predicts nucleotides 1 to 54 of SEQ ID NO: 15 encode a signal peptide. In another aspect, the mature polypeptide coding sequence is nucleotides 55 to 996 of SEQ ID NO: 17 based on the SignalP 3.0 program that predicts nucleotides 1 to 54 of SEQ ID NO: 17 encode a signal peptide. In another aspect, the mature polypeptide coding sequence is nucleotides 49 to 987 of SEQ ID NO: 19 based on the SignalP 3.0 program that predicts nucleotides 1 to 48 of SEQ ID NO: 19 encode a signal peptide. In another aspect, the mature polypeptide coding sequence is nucleotides 49 to 1236 of SEQ ID NO: 21 based on the SignalP 3.0 program that predicts nucleotides 1 to 48 of SEQ ID NO: 21 encode a signal peptide. In another aspect, the mature polypeptide coding sequence is nucleotides 55 to 996 of SEQ ID NO: 23 based on the SignalP 3.0 program that predicts nucleotides 1 to 54 of SEQ ID NO: 23 encode a signal peptide. In another aspect, the mature polypeptide coding sequence is nucleotides 49 to 1182 of SEQ ID NO: 25 based on the SignalP 3.0 program that predicts nucleotides 1 to 48 of SEQ ID NO: 25 encode a signal peptide. In another aspect, the mature polypeptide coding sequence is nucleotides 55 to 987 of SEQ ID NO: 27 based on the SignalP 3.0 program that predicts nucleotides 1 to 54 of SEQ ID NO: 27 encode a signal peptide. In another aspect, the mature polypeptide coding sequence is nucleotides 55 to 1403 of SEQ ID NO: 29 based on the SignalP 3.0 program that predicts nucleotides 1 to 54 of SEQ ID NO: 29 encode a signal peptide. In another aspect, the mature polypeptide coding sequence is nucleotides 58 to 1230 of SEQ ID NO: 31 based on the SignalP 3.0 program that predicts nucleotides 1 to 57 of SEQ ID NO: 31 encode a signal peptide. In another aspect, the mature polypeptide coding sequence is nucleotides 55 to 996 of SEQ ID NO: 33 based on the SignalP 3.0 program that predicts nucleotides 1 to 54 of SEQ ID NO: 33 encode a signal peptide. In another aspect, the mature polypeptide coding sequence is nucleotides 52 to 999 of SEQ ID NO: 35 based on the SignalP 3.0 program that predicts nucleotides 1 to 51 of SEQ ID NO: 35 encode a signal peptide. In another aspect, the mature polypeptide coding sequence is nucleotides 58 to 1225 of SEQ ID NO: 37 based on the SignalP 3.0 program that predicts nucleotides 1 to 57 of SEQ ID NO: 37 encode a signal peptide. In another aspect, the mature polypeptide coding sequence is nucleotides 58 to 1167 of SEQ ID NO: 39 based on the SignalP 3.0 program that predicts nucleotides 1 to 57 of SEQ ID NO: 39 encode a signal peptide. The term “mature polypeptide coding sequence” is understood herein to also include the genomic DNA sequence or the cDNA sequence of the specified coding sequence.

Mutant: The term “mutant” means a polynucleotide encoding a variant.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Parent or parent endoglucanase: The term “parent” or “parent endoglucanase” means an endoglucanase to which an alteration, i.e., a substitution, insertion, and/or deletion, is made at one or more (e.g., several) positions to produce the endoglucanase variants of the present invention. The parent may be a naturally occurring (wild-type) polypeptide or a variant or fragment thereof.

Peroxidase: The term “peroxidase” means an enzyme that converts a peroxide, e.g., hydrogen peroxide, to a less oxidative species, e.g., water. It is understood herein that a peroxidase encompasses a peroxide-decomposing enzyme. The term “peroxide-decomposing enzyme” is defined herein as an donor:peroxide oxidoreductase (E.C. number 1.11.1.x, wherein x=1-3, 5, 7-19, or 21) that catalyzes the reaction reduced substrate (2e⁻)+ROOR′→oxidized substrate+ROH+R′OH; such as horseradish peroxidase that catalyzes the reaction phenol+H₂O₂→quinone+H₂O, and catalase that catalyzes the reaction H₂O₂+H₂O₂→O₂+2H₂O. In addition to hydrogen peroxide, other peroxides may also be decomposed by these enzymes.

Pretreated cellulosic or hemicellulosic material: The term “pretreated cellulosic or hemicellulosic material” means a cellulosic or hemicellulosic material derived from biomass by treatment with heat and dilute sulfuric acid, alkaline pretreatment, neutral pretreatment, or any pretreatment known in the art.

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are a gap open penalty of 10, a gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having endoglucanase activity. In one aspect, a subsequence contains at least 85%, at least 90%, or at least 95% of the nucleotides of the mature polypeptide coding sequence.

Variant: The term “variant” means a polypeptide having endoglucanase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95%, or at least 100% of the endoglucanase activity of the parent endoglucanase.

Wild-type endoglucanase: The term “wild-type” endoglucanase means an endoglucanase expressed by a naturally occurring microorganism, such as a bacterium, yeast, or filamentous fungus found in nature.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrimann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylan degrading activity can be determined by measuring the increase in hydrolysis of birchwood xylan (Sigma Chemical Co., Inc.) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### Conventions for Designation of Variants

For purposes of the present invention, the full-length polypeptide disclosed in SEQ ID NO: 2 is used to determine the corresponding amino acid residue in another endoglucanase. The amino acid sequence of another endoglucanase is aligned with the full-length polypeptide disclosed in SEQ ID NO: 2, and based on the alignment, the amino acid position number corresponding to any amino acid residue in the full-length polypeptide of SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. Numbering of the amino acid positions is based on the full-length polypeptide (e.g., including the signal peptide) of SEQ ID NO: 2 wherein position 1 is the first amino acid of the signal peptide (i.e., Met) and position 31 is the first amino acid (i.e., Ala) of the mature polypeptide of SEQ ID NO: 2.

Identification of the corresponding amino acid residue in another endoglucanase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When another endoglucanase has diverged from the full-length polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the endoglucanase variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions.

For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Different Alterations.

Where different alterations can be introduced at a position, the different alterations are separated by a comma, e.g., “Arg170Tyr,Glu” or “R170Y,E” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates to isolated endoglucanase variants, comprising a substitution at one or more (e.g., several) positions corresponding to positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2, wherein the variant has endoglucanase activity.

### Variants

In an embodiment, the variants have a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, to the amino acid sequence of the parent endoglucanase.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the polypeptide of SEQ ID NO: 2, 4, 6, 8, 10, 12, 14, 16, 18, 20, 22, 24, 26, 28, 30, 32, 34, 36, 38, or 40.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 2, 4, 6, 8, 10, 12, 14, 16, 18, 20, 22, 24, 26, 28, 30, 32, 34, 36, 38, or 40.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 2.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 4.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 6.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 8.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 10.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 12.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 14.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 16.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 18.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 20.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 22.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 24.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 26.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 28.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 30.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 32.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 34.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 36.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 38.

In another embodiment, the variants have at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100%, sequence identity to the mature polypeptide of SEQ ID NO: 40.

In one aspect, the number of substitutions in the variants of the present invention is 1-23, e.g., 1, 2, 3, 4, 5, 6, 7, 8, 9, 10, 11, 12, 13, 14, 15, 16, 17, 18, 19, 20, 21, 22, or 23 substitutions.

In another aspect, a variant comprises a substitution at one or more (e.g., several) positions corresponding to positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at two positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at three positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at four positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at five positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at six positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at seven positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at eight positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at nine positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at ten positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at eleven positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at twelve positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at thirteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at fourteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at fifteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at sixteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at seventeen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at eighteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at nineteen positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at twenty positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at twenty-one positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at twenty-two positions corresponding to any of positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2. In another aspect, a variant comprises a substitution at each position corresponding to positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2.

In another aspect, the variant comprises or consists of a substitution at a position corresponding to position 43. In another aspect, the amino acid at a position corresponding to position 43 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ser. In another aspect, the variant comprises or consists of the substitution G43S of the mature polypeptide of SEQ ID NO: 2.

In another aspect, the variant comprises or consists of a substitution at a position corresponding to position 49. In another aspect, the amino acid at a position corresponding to position 49 is substituted with Ala, Arg, Asn, Asp, Cys, Gin, Glu, Gly, His, Ile, Leu, Lys, Met, Phe, Pro, Ser, Thr, Trp, Tyr, or Val, preferably with His. In another aspect, the variant comprises or consists of the substitution Q49H of the mature polypeptide of SEQ ID NO: 2.

In another aspect, the variant comprises or consists of a substitution at a position corresponding to position 51. In another aspect, the amino acid at a position corresponding to position 51 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant comprises or consists of the substitution L51R of the mature polypeptide of SEQ ID NO: 2.

In another aspect, the variant comprises or consists of a substitution at a position corresponding to position 55. In another aspect, the amino acid at a position corresponding to position 55 is substituted with Ala, Arg, Asn, Asp, Cys, Gin, Glu, Gly, His, Ile, Leu, Lys, Met, Phe, Pro, Ser, Thr, Trp, Tyr, or Val, preferably with Lys. In another aspect, the variant comprises or consists of the substitution E55K of the mature polypeptide of SEQ ID NO: 2.

In another aspect, the variant comprises or consists of a substitution at a position corresponding to position 63. In another aspect, the amino acid at a position corresponding to position 63 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu, His, or Asn. In another aspect, the variant comprises or consists of the substitution D63E,H,N of the mature polypeptide of SEQ ID NO: 2.

In another aspect, the variant comprises or consists of a substitution at a position corresponding to position 72. In another aspect, the amino acid at a position corresponding to position 72 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant comprises or consists of the substitution S72R of the mature polypeptide of SEQ ID NO: 2.

In another aspect, the variant comprises or consists of a substitution at a position corresponding to position 102. In another aspect, the amino acid at a position corresponding to position 102 is substituted with Ala, Arg, Asn, Asp, Cys, Gin, Glu, Gly, His, Ile, Leu, Lys, Met, Phe, Pro, Ser, Thr, Trp, Tyr, or Val, preferably with His. In another aspect, the variant comprises or consists of the substitution A102T of the mature polypeptide of SEQ ID NO: 2.

In another aspect, the variant comprises or consists of a substitution at a position corresponding to position 103. In another aspect, the amino acid at a position corresponding to position 103 is substituted with Ala, Arg, Asn, Asp, Cys, Gin, Glu, Gly, His, Ile, Leu, Lys, Met, Phe, Pro, Ser, Thr, Trp, Tyr, or Val, preferably with Gly. In another aspect, the variant comprises or consists of the substitution D103G of the mature polypeptide of SEQ ID NO: 2.

In another aspect, the variant comprises or consists of a substitution at a position corresponding to position 109. In another aspect, the amino acid at a position corresponding to position 109 is substituted with Ala, Arg, Asn, Asp, Cys, Gin, Glu, Gly, His, Ile, Leu, Lys, Met, Phe, Pro, Ser, Thr, Trp, Tyr, or Val, preferably with Lys. In another aspect, the variant comprises or consists of the substitution N109K of the mature polypeptide of SEQ ID NO: 2.

In another aspect, the variant comprises or consists of a substitution at a position corresponding to position 137. In another aspect, the amino acid at a position corresponding to position 137 is substituted with Ala, Arg, Asn, Asp, Cys, Gin, Glu, Gly, His, Ile, Leu, Lys, Met, Phe, Pro, Ser, Thr, Trp, Tyr, or Val, preferably with Thr. In another aspect, the variant comprises or consists of the substitution S137T of the mature polypeptide of SEQ ID NO: 2.

In another aspect, the variant comprises or consists of a substitution at a position corresponding to position 141. In another aspect, the amino acid at a position corresponding to position 141 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ala. In another aspect, the variant comprises or consists of the substitution T141A of the mature polypeptide of SEQ ID NO: 2.

In another aspect, the variant comprises or consists of a substitution at a position corresponding to position 148. In another aspect, the amino acid at a position corresponding to position 148 is substituted with Ala, Arg, Asn, Asp, Cys, Gin, Glu, Gly, His, Ile, Leu, Lys, Met, Phe, Pro, Ser, Thr, Trp, Tyr, or Val, preferably with Thr. In another aspect, the variant comprises or consists of the substitution S148T of the mature polypeptide of SEQ ID NO: 2.

In another aspect, the variant comprises or consists of a substitution at a position corresponding to position 149. In another aspect, the amino acid at a position corresponding to position 149 is substituted with Ala, Arg, Asn, Asp, Cys, Gin, Glu, Gly, His, Ile, Leu, Lys, Met, Phe, Pro, Ser, Thr, Trp, Tyr, or Val, preferably with His. In another aspect, the variant comprises or consists of the substitution Q149H of the mature polypeptide of SEQ ID NO: 2.

In another aspect, the variant comprises or consists of a substitution at a position corresponding to position 179. In another aspect, the amino acid at a position corresponding to position 179 is substituted with Ala, Arg, Asn, Asp, Cys, Gin, Glu, Gly, His, Ile, Leu, Lys, Met, Phe, Pro, Ser, Thr, Trp, Tyr, or Val, preferably with Thr. In another aspect, the variant comprises or consists of the substitution A179T of the mature polypeptide of SEQ ID NO: 2.

In another aspect, the variant comprises or consists of a substitution at a position corresponding to position 211. In another aspect, the amino acid at a position corresponding to position 211 is substituted with Ala, Arg, Asn, Asp, Cys, Gin, Glu, Gly, His, Ile, Leu, Lys, Met, Phe, Pro, Ser, Thr, Trp, Tyr, or Val, preferably with His. In another aspect, the variant comprises or consists of the substitution D211H of the mature polypeptide of SEQ ID NO: 2.

In another aspect, the variant comprises or consists of a substitution at a position corresponding to position 215. In another aspect, the amino acid at a position corresponding to position 215 is substituted with Ala, Arg, Asn, Asp, Cys, Gin, Glu, Gly, His, Ile, Leu, Lys, Met, Phe, Pro, Ser, Thr, Trp, Tyr, or Val, preferably with Ile. In another aspect, the variant comprises or consists of the substitution S215I of the mature polypeptide of SEQ ID NO: 2.

In another aspect, the variant comprises or consists of a substitution at a position corresponding to position 240. In another aspect, the amino acid at a position corresponding to position 240 is substituted with Ala, Arg, Asn, Asp, Cys, Gin, Glu, Gly, His, Ile, Leu, Lys, Met, Phe, Pro, Ser, Thr, Trp, Tyr, or Val, preferably with Val. In another aspect, the variant comprises or consists of the substitution A240V of the mature polypeptide of SEQ ID NO: 2.

In another aspect, the variant comprises or consists of a substitution at a position corresponding to position 250. In another aspect, the amino acid at a position corresponding to position 250 is substituted with Ala, Arg, Asn, Asp, Cys, Gin, Glu, Gly, His, Ile, Leu, Lys, Met, Phe, Pro, Ser, Thr, Trp, Tyr, or Val, preferably with Gin. In another aspect, the variant comprises or consists of the substitution E250Q of the mature polypeptide of SEQ ID NO: 2.

In another aspect, the variant comprises or consists of a substitution at a position corresponding to position 254. In another aspect, the amino acid at a position corresponding to position 254 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant comprises or consists of the substitution S254R of the mature polypeptide of SEQ ID NO: 2.

In another aspect, the variant comprises or consists of a substitution at a position corresponding to position 277. In another aspect, the amino acid at a position corresponding to position 277 is substituted with Ala, Arg, Asn, Asp, Cys, Gin, Glu, Gly, His, Ile, Leu, Lys, Met, Phe, Pro, Ser, Thr, Trp, Tyr, or Val, preferably with Asn or Val. In another aspect, the variant comprises or consists of the substitution D277N,V of the mature polypeptide of SEQ ID NO: 2.

In another aspect, the variant comprises or consists of a substitution at a position corresponding to position 284. In another aspect, the amino acid at a position corresponding to position 284 is substituted with Ala, Arg, Asn, Asp, Cys, Gin, Glu, Gly, His, Ile, Leu, Lys, Met, Phe, Pro, Ser, Thr, Trp, Tyr, or Val, preferably with Arg. In another aspect, the variant comprises or consists of the substitution T284R of the mature polypeptide of SEQ ID NO: 2.

In another aspect, the variant comprises or consists of a substitution at a position corresponding to position 288. In another aspect, the amino acid at a position corresponding to position 288 is substituted with Ala, Arg, Asn, Asp, Cys, Gin, Glu, Gly, His, Ile, Leu, Lys, Met, Phe, Pro, Ser, Thr, Trp, Tyr, or Val, preferably with Glu, Val, or Tyr. In another aspect, the variant comprises or consists of the substitution D288E,V,Y of the mature polypeptide of SEQ ID NO: 2.

In another aspect, the variant comprises or consists of a substitution at a position corresponding to position 320. In another aspect, the amino acid at a position corresponding to position 320 is substituted with Ala, Arg, Asn, Asp, Cys, Gin, Glu, Gly, His, Ile, Leu, Lys, Met, Phe, Pro, Ser, Thr, Trp, Tyr, or Val, preferably with His or Lys. In another aspect, the variant comprises or consists of the substitution N320H,K of the mature polypeptide of SEQ ID NO: 2.

In each of the aspects above, the variant comprises or consists of one or more substitutions described above at positions corresponding to the full-length polypeptide of SEQ ID NO: 2 in other endoglucanases as parents.

In each of the aspects below, the variant comprises or consists of one or more substitutions described below at positions corresponding to the full-length polypeptide of SEQ ID NO: 2 in other endoglucanases described herein or at positions of the full-length polypeptide of SEQ ID NO: 2.

In another aspect, the variant comprises or consists of one or more (e.g., several) substitutions selected from the group consisting of G43S; Q49H; L51R; E55K; D63E,H,N; S72R; A102T; D103G; N109K; S137T; T141A; S148T; Q149H; A179T; D211H; S215I; A240V; E250Q; S254R; D277N,V; T284R; D288E,V,Y; and N320H,K.

In another aspect, the variant comprises or consists of the substitutions D63H+E250Q of the mature polypeptide of SEQ ID NO: 2.

In another aspect, the variant comprises or consists of the substitutions T284R+D288V of the mature polypeptide of SEQ ID NO: 2.

In another aspect, the variant comprises or consists of the substitutions L51R+D288Y of the mature polypeptide of SEQ ID NO: 2.

In another aspect, the variant comprises or consists of the substitutions D63H+Q149H+S254R of the mature polypeptide of SEQ ID NO: 2.

In another aspect, the variant comprises or consists of the substitutions D63E+A240V+D277N+N320K of the mature polypeptide of SEQ ID NO: 2.

In another aspect, the variant comprises or consists of the substitutions E55K+D63N+D277V+D288E of the mature polypeptide of SEQ ID NO: 2.

In another aspect, the variant comprises or consists of the substitutions A102T+D211H+S215I+N320H of the mature polypeptide of SEQ ID NO: 2.

In another aspect, the variant comprises or consists of the substitutions Q49H+E55K+S148T+D277V of the mature polypeptide of SEQ ID NO: 2.

In another aspect, the variant comprises or consists of the substitutions D63N+D103G+N109K+D277V of the mature polypeptide of SEQ ID NO: 2.

In another aspect, the variant comprises or consists of the substitutions E55K+A179T+S254R+D288E+N320K of the mature polypeptide of SEQ ID NO: 2.

In another aspect, the variant comprises or consists of the substitutions G43S+E55K+D63N+S72R+S137T+T141A of the mature polypeptide of SEQ ID NO: 2.

The variants may further comprise one or more additional alterations, e.g., substitutions, insertions, and/or deletions at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/lle, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for endoglucanase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The variants may consist of 270 to 317 amino acids, e.g., 270 to 275, 276 to 280, 281 to 285, 286 to 290, 291 to 295, 296 to 300, 301 to 305, 306 to 310, and 311 to 317 amino acids.

In an embodiment, the variant has increased specific performance compared to the parent enzyme of at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold.

### Parent Endoglucanases

The parent endoglucanase may be any endoglucanase. The parent endoglucanase may be (a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, or 40; (b) a polypeptide encoded by a polynucleotide that hybridizes under low stringency conditions with the full-length complement of the mature polypeptide coding sequence of SEQ ID NO: 1, 3, 5, 7, 9, 11, 13, 15, 17, 19, 21, 23, 25, 27, 29, 31, 33, 35, 37, or 39, or (c) a polypeptide encoded by a polynucleotide having at least 60% sequence identity to the mature polypeptide coding sequence of SEQ ID NO: 1, 3, 5, 7, 9, 11, 13, 15, 17, 19, 21, 23, 25, 27, 29, 31, 33, 35, 37, or 39.

In one aspect, the parent has a sequence identity to the mature polypeptide of SEQ ID NO: 2, 4, 6, 8, 10, 12, 14, 16, 18, 20, 22, 24, 26, 28, 30, 32, 34, 36, 38, or 40 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have endoglucanase activity. In one aspect, the amino acid sequence of the parent differs by up to 10 amino acids, e.g., 1, 2, 3, 4, 5, 6, 7, 8, 9, or 10, from the mature polypeptide of SEQ ID NO: 2, 4, 6, 8, 10, 12, 14, 16, 18, 20, 22, 24, 26, 28, 30, 32, 34, 36, 38, or 40.

In another aspect, the parent comprises or consists of the amino acid sequence of SEQ ID NO: 2, 4, 6, 8, 10, 12, 14, 16, 18, 20, 22, 24, 26, 28, 30, 32, 34, 36, 38, or 40. In another aspect, the parent comprises or consists of the mature polypeptide of SEQ ID NO: 2, 4, 6, 8, 10, 12, 14, 16, 18, 20, 22, 24, 26, 28, 30, 32, 34, 36, 38, or 40.

In another aspect, the parent is a fragment of the mature polypeptide of SEQ ID NO: 2, 4, 6, 8, 10, 12, 14, 16, 18, 20, 22, 24, 26, 28, 30, 32, 34, 36, 38, or 40 containing at least 85%, e.g., at least 90% and at least 95% of the amino acid residues.

In another embodiment, the parent is an allelic variant of the mature polypeptide of SEQ ID NO: 2, 4, 6, 8, 10, 12, 14, 16, 18, 20, 22, 24, 26, 28, 30, 32, 34, 36, 38, or 40.

In another aspect, the parent is encoded by a polynucleotide that hybridizes under very low stringency conditions, low stringency conditions, medium stringency conditions, medium-high stringency conditions, high stringency conditions, or very high stringency conditions with the full-length complement of the mature polypeptide coding sequence of SEQ ID NO: 1, 3, 5, 7, 9, 11, 13, 15, 17, 19, 21, 23, 25, 27, 29, 31, 33, 35, 37, or 39 (Sambrook et al., 1989, Molecular Cloning, A Laboratory Manual, 2d edition, Cold Spring Harbor, N.Y.).

The polynucleotide of SEQ ID NO: 1, 3, 5, 7, 9, 11, 13, 15, 17, 19, 21, 23, 25, 27, 29, 31, 33, 35, 37, or 39, or a subsequence thereof, as well as the polypeptide of SEQ ID NO: 2, 4, 6, 8, 10, 12, 14, 16, 18, 20, 22, 24, 26, 28, 30, 32, 34, 36, 38, or 40, or a fragment thereof, may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, 3, 5, 7, 9, 11, 13, 15, 17, 19, 21, 23, 25, 27, 29, 31, 33, 35, 37, or 39 or a subsequence thereof, the carrier material is used in a Southern blot.

For purposes of the present invention, hybridization indicates that the polynucleotide hybridizes to a labeled nucleic acid probe corresponding to the full-length complement of (i) SEQ ID NO: 1, 3, 5, 7, 9, 11, 13, 15, 17, 19, 21, 23, 25, 27, 29, 31, 33, 35, 37, or 39; (ii) the mature polypeptide coding sequence of SEQ ID NO: 1, 3, 5, 7, 9, 11, 13, 15, 17, 19, 21, 23, 25, 27, 29, 31, 33, 35, 37, or 39; or (iii) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of SEQ ID NO: 1, 3, 5, 7, 9, 11, 13, 15, 17, 19, 21, 23, 25, 27, 29, 31, 33, 35, 37, or 39. In another aspect, the nucleic acid probe is a polynucleotide that encodes the polypeptide of SEQ ID NO: 2, 4, 6, 8, 10, 12, 14, 16, 18, 20, 22, 24, 26, 28, 30, 32, 34, 36, 38, or 40; the mature polypeptide thereof; or a fragment thereof. In another aspect, the nucleic acid probe is SEQ ID NO: 1, 3, 5, 7, 9, 11, 13, 15, 17, 19, 21, 23, 25, 27, 29, 31, 33, 35, 37, or 39.

In another embodiment, the parent is encoded by a polynucleotide having a sequence identity to the mature polypeptide coding sequence of SEQ ID NO: 1, 3, 5, 7, 9, 11, 13, 15, 17, 19, 21, 23, 25, 27, 29, 31, 33, 35, 37, or 39 of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The parent may be a hybrid polypeptide (chimera) in which a region of the parent is replaced with a region of another polypeptide.

The parent may be a fusion polypeptide or cleavable fusion polypeptide in which another polypeptide is fused at the N-terminus or the C-terminus of the parent. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

In one aspect, the parent is a yeast endoglucanase. In another aspect, the parent is a filamentous fungal endoglucanase.

In another aspect, the parent is an Aspergillus (Emericella) endoglucanase. In another aspect, the parent is an Aspergillus aculeatus endoglucanase. In another aspect, the parent is an Aspergillus awamori endoglucanase. In another aspect, the parent is an Aspergillus clavatus endoglucanase. In another aspect, the parent is an Aspergillus fumigatus endoglucanase. In another aspect, the parent is an Aspergillus kawachii endoglucanase. In another aspect, the parent is an Aspergillus nidulans endoglucanase. In another aspect, the parent is an Aspergillus oryzae endoglucanase.

In another aspect, the parent is a Chaetomium endoglucanase. In another aspect, the parent is a Chaetomium virescens endoglucanase.

In another aspect, the parent is a Myceliophthora endoglucanase. In another aspect, the parent is a Myceliophthora thermophila endoglucanase.

In another aspect, the parent is a Neosartorya endoglucanase. In another aspect, the parent is a Neosartorya fischeri endoglucanase.

In another aspect, the parent is a Penicillium endoglucanase. In another aspect, the parent is a Penicillium emersonii endoglucanase. In another aspect, the parent is a Penicillium spinulosum endoglucanase.

In another aspect, the parent is a Talaromyces endoglucanase. In another aspect, the parent is a Talaromyces emersonii endoglucanase. In another aspect, the parent is a Talaromyces leycettanus endoglucanase. In another aspect, the parent is a Talaromyces pinophilus endoglucanase.

In another aspect, the parent is a Thermoascus endoglucanase. In another aspect, the parent is a Thermoascus aurantiacus endoglucanase.

In another aspect, the parent is a Trichoderma endoglucanase. In another aspect, the parent is a Trichoderma reesei endoglucanase.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding a parent may then be obtained by similarly screening a genomic DNA or cDNA library of another microorganism or mixed DNA sample. Once a polynucleotide encoding a parent has been detected with the probe(s), the polynucleotide can be isolated or cloned by utilizing techniques that are known to those of ordinary skill in the art (see, e.g., Sambrook et al., 1989, supra).

### Preparation of Variants

The present invention also relates to methods for obtaining a variant having endoglucanase activity, comprising: (a) introducing into a parent endoglucanase a substitution at one or more (e.g., several) positions corresponding to positions 43, 49, 51, 55, 63, 72, 102, 103, 109, 137, 141, 148, 149, 179, 211, 215, 240, 250, 254, 277, 284, 288, and 320 of the full-length polypeptide of SEQ ID NO: 2, wherein the variant has endoglucanase activity; and optionally (b) recovering the variant.

The variants can be prepared using any mutagenesis procedure known in the art, such as site-directed mutagenesis, site-saturation mutagenesis, synthetic gene construction, semi-synthetic gene construction, random mutagenesis, shuffling, etc.

The variants can be prepared using any mutagenesis procedure known in the art, such as site-directed mutagenesis, site-saturation mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Site-saturation mutagenesis systematically replaces a polypeptide coding sequence with sequences encoding all 19 amino acids at one or more (e.g., several) specific positions (Parikh and Matsumura, 2005, J. Mol. Biol. 352: 621-628).

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### Polynucleotides

The present invention also relates to isolated polynucleotides encoding a variant of the present invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIII/A gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the variant. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked to the regulatory sequence.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

### Host Cells

The present invention also relates to recombinant host cells, comprising a polynucleotide encoding a variant of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp. 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising (a) cultivating a recombinant host cell of the present invention under conditions conducive for production of the variant; and optionally (b) recovering the variant.

The host cells are cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variants may be detected using methods known in the art that are specific for the endoglucanases. These detection methods include, but are not limited to, use of specific antibodies, formation of an enzyme product, or disappearance of an enzyme substrate. For example, an enzyme assay may be used to determine the activity of the variant, as described herein.

The variant may be recovered using methods known in the art. For example, the variant may be recovered from the nutrient medium by conventional procedures including, but not limited to, collection, centrifugation, filtration, extraction, spray-drying, evaporation, or precipitation. In one aspect, the whole fermentation broth is recovered.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

### Fermentation Broth Formulations or Cell Compositions

The present invention also relates to fermentation broth formulations or cell compositions comprising a variant of the present invention. The fermentation broth products further comprise additional ingredients used in the fermentation process, such as, for example, cells (including, the host cells containing the gene encoding the variant of the present invention which are used to produce the variant of interest), cell debris, biomass, fermentation media and/or fermentation products. In some embodiments, the compositions are cell-killed whole broths containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulations and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In another embodiment, the compositions contain an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The fermentation broth formulations or cell compositions may further comprise multiple enzymatic activities, such as one or more (e.g., several) enzymes selected from the group consisting of a cellulase, a hemicellulase, an esterase, an expansin, a laccase, a ligninolytic enzyme, a pectinase, a peroxidase, a protease, and a swollenin. The fermentation broth formulations or cell compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The cell-killed whole broths or compositions may contain the unfractionated contents of the fermentation materials derived at the end of the fermentation. Typically, the cell-killed whole broths or compositions contains the spent culture medium and cell debris present after the microbial cells (e.g., filamentous fungal cells) are grown to saturation, incubated under carbon-limiting conditions to allow protein synthesis. In some embodiments, the cell-killed whole broths or compositions contain the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a variant of the present invention. Preferably, the compositions are enriched in such a variant. The term “enriched” indicates that the endoglucanase activity of the composition has been increased, e.g., with an enrichment factor of at least 1.1.

The compositions may comprise a variant of the present invention as the major enzymatic component, e.g., a mono-component composition. Alternatively, the compositions may comprise multiple enzymatic activities, such as one or more (e.g., several) enzymes selected from the group consisting of a cellulase, a hemicellulase, an AA9 polypeptide having cellulolytic enhancing activity, an esterase, an expansin, a laccase, a ligninolytic enzyme, a pectinase, a peroxidase, a protease, and a swollenin. The compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The present invention is also directed to the following processes for using the endoglucanase variants, or compositions thereof.

The present invention also relates to processes for degrading a cellulosic material, comprising: treating the cellulosic material with an enzyme composition comprising an endoglucanase variant of the present invention. In one aspect, the processes further comprise recovering the degraded cellulosic material. Soluble products from the degradation of the cellulosic material can be separated from insoluble cellulosic material using methods known in the art such as, for example, centrifugation, filtration, or gravity settling.

The present invention also relates to processes of producing a fermentation product, comprising: (a) saccharifying a cellulosic material with an enzyme composition comprising an endoglucanase variant of the present invention; (b) fermenting the saccharified cellulosic material with one or more (e.g., several) fermenting microorganisms to produce the fermentation product; and (c) recovering the fermentation product from the fermentation.

The present invention also relates to processes of fermenting a cellulosic material, comprising: fermenting the cellulosic material with one or more (e.g., several) fermenting microorganisms, wherein the cellulosic material is saccharified with an enzyme composition comprising an endoglucanase variant of the present invention. In one aspect, the fermenting of the cellulosic material produces a fermentation product. In another aspect, the processes further comprise recovering the fermentation product from the fermentation.

The processes of the present invention can be used to saccharify the cellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic material typically involves pretreatment, enzymatic hydrolysis (saccharification), and fermentation.

The processing of the cellulosic material according to the present invention can be accomplished using methods conventional in the art. Moreover, the processes of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing the processes of the present invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

Pretreatment.

In practicing the processes of the present invention, any pretreatment process known in the art can be used to disrupt plant cell wall components of the cellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the cellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one aspect, the chemical pretreatment is preferably carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt % acid, e.g., 0.05 to 5 wt % acid or 0.1 to 2 wt % acid. The acid is contacted with the cellulosic material and held at a temperature in the range of preferably 140-200° C., e.g., 165-190° C., for periods ranging from 1 to 60 minutes.

In another aspect, pretreatment takes place in an aqueous slurry. In preferred aspects, the cellulosic material is present during pretreatment in amounts preferably between 10-80 wt. %, e.g., 20-70 wt. % or 30-60 wt. %, such as around 40 wt. %. The pretreated cellulosic material can be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in a preferred aspect, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

Saccharification.

In the hydrolysis step, also known as saccharification, the cellulosic material, e.g., pretreated, is hydrolyzed to break down cellulose and/or hemicellulose to fermentable sugars, such as glucose, cellobiose, xylose, xylulose, arabinose, mannose, galactose, and/or soluble oligosaccharides. The hydrolysis is performed enzymatically by one or more enzyme compositions in one or more stages. The hydrolysis can be carried out as a batch process or series of batch processes. The hydrolysis can be carried out as a fed batch or continuous process, or series of fed batch or continuous processes, where the cellulosic or hemicellulosic material is fed gradually to, for example, a hydrolysis solution containing an enzyme composition. In an embodiment the saccharification is a continuous saccharification in which a cellulosic material and a cellulolytic enzyme composition are added at different intervals throughout the saccharification and the hydrolysate is removed at different intervals throughout the saccharification. The removal of the hydrolysate may occur prior to, simultaneously with, or after the addition of the cellulosic material and the cellulolytic enzyme composition.

Enzymatic hydrolysis is preferably carried out in a suitable aqueous environment under conditions that can be readily determined by one skilled in the art. In one aspect, hydrolysis is performed under conditions suitable for the activity of the enzymes(s), i.e., optimal for the enzyme(s).

The saccharification is generally performed in stirred-tank reactors or fermentors under controlled pH, temperature, and mixing conditions. Suitable process time, temperature and pH conditions can readily be determined by one skilled in the art. For example, the total saccharification time can last up to 200 hours, but is typically performed for preferably about 4 to about 120 hours, e.g., about 12 to about 96 hours or about 24 to about 72 hours. The temperature is in the range of preferably about 25° C. to about 80° C., e.g., about 30° C. to about 70° C., about 40° C. to about 60° C., or about 50° C. to about 55° C. The pH is in the range of preferably about 3 to about 9, e.g., about 3.5 to about 8, about 4 to about 7, about 4.2 to about 6, or about 4.3 to about 5.5.

The dry solids content is in the range of preferably about 5 to about 50 wt. %, e.g., about 10 to about 40 wt. % or about 20 to about 30 wt. %.

In one aspect, the saccharification is performed in the presence of dissolved oxygen at a concentration of at least 0.5% of the saturation level.

In an embodiment of the invention the dissolved oxygen concentration during saccharification is in the range of at least 0.5% up to 30% of the saturation level, such as at least 1% up to 25%, at least 1% up to 20%, at least 1% up to 15%, at least 1% up to 10%, at least 1% up to 5%, and at least 1% up to 3%. In a preferred embodiment, the dissolved oxygen concentration is maintained at a concentration of at least 0.5% up to 30% of the saturation level, such as at least 1% up to 25%, at least 1% up to 20%, at least 1% up to 15%, at least 1% up to 10%, at least 1% up to 5%, and at least 1% up to 3% during at least 25%, such as at least 50% or at least 75% of the saccharification period. When the enzyme composition comprises an oxidoreductase the dissolved oxygen concentration may be higher up to 70% of the saturation level.

Oxygen is added to the vessel in order to achieve the desired concentration of dissolved oxygen during saccharification. Maintaining the dissolved oxygen level within a desired range can be accomplished by aeration of the vessel, tank or the like by adding compressed air through a diffuser or sparger, or by other known methods of aeration. The aeration rate can be controlled on the basis of feedback from a dissolved oxygen sensor placed in the vessel/tank, or the system can run at a constant rate without feedback control. In the case of a hydrolysis train consisting of a plurality of vessels/tanks connected in series, aeration can be implemented in one or more or all of the vessels/tanks. Oxygen aeration systems are well known in the art. According to the invention any suitable aeration system may be used. Commercial aeration systems are designed by, e.g., Chemineer, Derby, England, and build by, e.g., Paul Mueller Company, MO, USA.

The enzyme compositions can comprise any protein useful in degrading the cellulosic material.

In one aspect, the enzyme composition comprises or further comprises one or more (e.g., several) proteins selected from the group consisting of a cellulase, an AA9 polypeptide, a hemicellulase, an esterase, an expansin, a ligninolytic enzyme, an oxidoreductase, a pectinase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another aspect, the oxidoreductase is preferably one or more (e.g., several) enzymes selected from the group consisting of a catalase, a laccase, and a peroxidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises an AA9 polypeptide. In another aspect, the enzyme composition comprises an endoglucanase and an AA9 polypeptide. In another aspect, the enzyme composition comprises a cellobiohydrolase and an AA9 polypeptide. In another aspect, the enzyme composition comprises a beta-glucosidase and an AA9 polypeptide. In another aspect, the enzyme composition comprises an endoglucanase and a cellobiohydrolase. In another aspect, the enzyme composition comprises an endoglucanase I, an endoglucanase II, or a combination of an endoglucanase I and an endoglucanase II, and a cellobiohydrolase I, a cellobiohydrolase II, or a combination of a cellobiohydrolase I and a cellobiohydrolase II. In another aspect, the enzyme composition comprises an endoglucanase and a beta-glucosidase. In another aspect, the enzyme composition comprises an endoglucanase I, an endoglucanase II, or a combination of an endoglucanase I and an endoglucanase II, and a beta-glucosidase. In another aspect, the enzyme composition comprises a beta-glucosidase and a cellobiohydrolase. In another aspect, the enzyme composition comprises a beta-glucosidase and a cellobiohydrolase I, a cellobiohydrolase II, or a combination of a cellobiohydrolase I and a cellobiohydrolase II. In another aspect, the enzyme composition comprises an endoglucanase, an AA9 polypeptide, and a cellobiohydrolase. In another aspect, the enzyme composition comprises an endoglucanase I, an endoglucanase II, or a combination of an endoglucanase I and an endoglucanase II, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and an AA9 polypeptide. In another aspect, the enzyme composition comprises a beta-glucosidase, an AA9 polypeptide, and a cellobiohydrolase. In another aspect, the enzyme composition comprises a beta-glucosidase, an AA9 polypeptide, and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a cellobiohydrolase. In another aspect, the enzyme composition comprises an endoglucanase I, an endoglucanase II, or a combination of an endoglucanase I and an endoglucanase II, a beta-glucosidase, and a cellobiohydrolase I, a cellobiohydrolase II, or a combination of a cellobiohydrolase I and a cellobiohydrolase II. In another aspect, the enzyme composition comprises an endoglucanase, a cellobiohydrolase, a beta-glucosidase, and an AA9 polypeptide. In another aspect, the enzyme composition comprises an endoglucanase I, an endoglucanase II, or a combination of an endoglucanase I and an endoglucanase II, a beta-glucosidase, an AA9 polypeptide, and a cellobiohydrolase I, a cellobiohydrolase II, or a combination of a cellobiohydrolase I and a cellobiohydrolase II.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In an embodiment, the xylanase is a Family 10 xylanase. In another embodiment, the xylanase is a Family 11 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a ligninolytic enzyme. In an embodiment, the ligninolytic enzyme is a manganese peroxidase. In another embodiment, the ligninolytic enzyme is a lignin peroxidase. In another embodiment, the ligninolytic enzyme is a H₂O₂-producing enzyme. In another aspect, the enzyme composition comprises a pectinase. In another aspect, the enzyme composition comprises an oxidoreductase. In an embodiment, the oxidoreductase is a catalase. In another embodiment, the oxidoreductase is a laccase. In another embodiment, the oxidoreductase is a peroxidase. In another aspect, the enzyme composition comprises a protease. In another aspect, the enzyme composition comprises a swollenin.

In the processes of the present invention, the enzyme(s) can be added prior to or during saccharification, saccharification and fermentation, or fermentation.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and/or native to the host cell. One or more (e.g., several) components of the enzyme composition may be produced as monocomponents, which are then combined to form the enzyme composition. The enzyme composition may be a combination of multicomponent and monocomponent protein preparations.

The enzymes used in the processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes and an endoglucanase variants depend on several factors including, but not limited to, the mixture of cellulolytic enzymes and/or hemicellulolytic enzymes, the cellulosic material, the concentration of cellulosic material, the pretreatment(s) of the cellulosic material, temperature, time, pH, and inclusion of a fermenting organism (e.g., for Simultaneous Saccharification and Fermentation).

In one aspect, an effective amount of cellulolytic or hemicellulolytic enzyme to the cellulosic material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic material.

In another aspect, an effective amount of an endoglucanase variant to the cellulosic material is about 0.01 to about 50.0 mg, e.g., about 0.01 to about 40 mg, about 0.01 to about 30 mg, about 0.01 to about 20 mg, about 0.01 to about 10 mg, about 0.01 to about 5 mg, about 0.025 to about 1.5 mg, about 0.05 to about 1.25 mg, about 0.075 to about 1.25 mg, about 0.1 to about 1.25 mg, about 0.15 to about 1.25 mg, or about 0.25 to about 1.0 mg per g of the cellulosic material.

In another aspect, an effective amount of an endoglucanase variant to cellulolytic or hemicellulolytic enzyme is about 0.005 to about 1.0 g, e.g., about 0.01 to about 1.0 g, about 0.15 to about 0.75 g, about 0.15 to about 0.5 g, about 0.1 to about 0.5 g, about 0.1 to about 0.25 g, or about 0.05 to about 0.2 g per g of cellulolytic or hemicellulolytic enzyme.

The polypeptides having cellulolytic enzyme activity or hemicellulolytic enzyme activity as well as other proteins/polypeptides useful in the degradation of the cellulosic material, e.g., AA9 polypeptides can be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, e.g., site-directed mutagenesis or shuffling.

Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host can be a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), SPEZYME™ CP (Genencor Int.), ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme preparation is added in an amount effective from about 0.001 to about 5.0 wt. % of solids, e.g., about 0.025 to about 4.0 wt. % of solids or about 0.005 to about 2.0 wt. % of solids.

Examples of bacterial endoglucanases that can be used in the processes of the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (GenBank:XM_324477), Humicola insolens endoglucanase V, Myceliophthora thermophila CBS 117.65 endoglucanase, Thermoascus aurantiacus endoglucanase I (GenBank:AF487830), Trichoderma reesei strain No. VTT-D-80133 endoglucanase (GenBank:M15665), and Penicillium pinophilum endoglucanase (WO 2012/062220).

Examples of cellobiohydrolases useful in the present invention include, but are not limited to, Aspergillus aculeatus cellobiohydrolase II (WO 2011/059740), Aspergillus fumigatus cellobiohydrolase I (WO 2013/028928), Aspergillus fumigatus cellobiohydrolase II (WO 2013/028928), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (GenBank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

In the processes of the present invention, any AA9 polypeptide can be used as a component of the enzyme composition.

Examples of AA9 polypeptides useful in the processes of the present invention include, but are not limited to, AA9 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290 and WO 2012/149344), Myceliophthora thermophila (WO 2009/085935, WO 2009/085859, WO 2009/085864, WO 2009/085868, and WO 2009/033071), Aspergillus fumigatus (WO 2010/138754), Penicillium pinophilum (WO 2011/005867), Thermoascus sp. (WO 2011/039319), Penicillium sp. (emersoni0 (WO 2011/041397 and WO 2012/000892), Thermoascus crustaceous (WO 2011/041504), Aspergillus aculeatus (WO 2012/125925), Thermomyces lanuginosus (WO 2012/113340, WO 2012/129699, WO 2012/130964, and WO 2012/129699), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206), and Talaromyces thermophilus (WO 2012/129697 and WO 2012/130950).

In one aspect, the AA9 polypeptide is used in the presence of a soluble activating divalent metal cation according to WO 2008/151043 or WO 2012/122518, e.g., manganese or copper.

In another aspect, the AA9 polypeptide is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

In one aspect, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one aspect, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes A/S), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Thermomyces lanuginosus (GeneSeqP:BAA22485), Talaromyces thermophilus (GeneSeqP:BAA22834), Thielavia terrestris NRRL 8126 (WO 2009/079210), and Trichophaea saccata (WO 2011/057083).

Examples of beta-xylosidases useful in the processes of the present invention include, but are not limited to, beta-xylosidases from Neurospora crassa (SwissProt: Q7SOW4), Trichoderma reesei (UniProtKB/TrEMBL:Q92458), Talaromyces emersonii (SwissProt: Q8X212), and Talaromyces thermophilus (GeneSeqP:BAA22816).

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (SwissProt:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Examples of oxidoreductases useful in the processes of the present invention include, but are not limited to, Aspergillus lentilus catalase, Aspergillus fumigatus catalase, Aspergillus niger catalase, Aspergillus oryzae catalase, Humicola insolens catalase, Neurospora crassa catalase, Penicillium emersonii catalase, Scytalidium thermophilum catalase, Talaromyces stipitatus catalase, Thermoascus aurantiacus catalase, Coprinus cinereus laccase, Myceliophthora thermophila laccase, Polyporus pinsitus laccase, Pycnoporus cinnabarinus laccase, Rhizoctonia solani laccase, Streptomyces coelicolor laccase, Coprinus cinereus peroxidase, Soy peroxidase, Royal palm peroxidase.

The polypeptides having enzyme activity used in the processes of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation.

The fermentable sugars obtained from the hydrolyzed cellulosic material can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous.

Any suitable hydrolyzed cellulosic material can be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Xylose fermenting yeast include strains of Candida, preferably C. sheatae or C. sonorensis; and strains of Pichia, e.g., P. stipitis, such as P. stipitis CBS 5773. Pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, G. P., 1996, Cellulose bioconversion technology, in Handbook on Bioethanol: Production and Utilization, Wyman, C. E., ed., Taylor & Francis, Washington, D.C., 179-212).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIO-FERM® AFT and XR (Lallemand Specialities, Inc., USA), ETHANOL RED® yeast (Lesaffre et Co,pagnie, France), FALI® (AB Mauri Food Inc., USA), FERMIOL® (Rymco International AG, Denmark), GERT STRAND™ (Gert Strand AB, Sweden), and SUPERSTART™ and THERMOSACC® fresh yeast (Lallemand Specialities, Inc., USA).

In an aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (co-fermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

In one aspect, the fermenting organism comprises a polynucleotide encoding an endoglucanase variant of the present invention.

In another aspect, the fermenting organism comprises one or more polynucleotides encoding one or more cellulolytic enzymes, hemicellulolytic enzymes, and accessory enzymes described herein.

It is well known in the art that the organisms described above can also be used to produce other substances, as described herein.

The fermenting microorganism is typically added to the degraded cellulosic material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded cellulosic material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation Products:

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one aspect, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another aspect, the fermentation product is an alkane. The alkane may be an unbranched or a branched alkane. The alkane can be, but is not limited to, pentane, hexane, heptane, octane, nonane, decane, undecane, or dodecane.

In another aspect, the fermentation product is a cycloalkane. The cycloalkane can be, but is not limited to, cyclopentane, cyclohexane, cycloheptane, or cyclooctane.

In another aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another aspect, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another aspect, the fermentation product is isoprene.

In another aspect, the fermentation product is a ketone. The term “ketone” encompasses a substance that contains one or more ketone moieties. The ketone can be, but is not limited to, acetone.

In another aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another aspect, the fermentation product is polyketide.

Recovery.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### Plants

The present invention also relates to isolated plants, e.g., a transgenic plant, plant part, or plant cell, comprising a polynucleotide of the present invention so as to express and produce the variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed (Sticklen, 2008, Nature Reviews 9: 433-443). For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

The present invention may be further described by the following numbered paragraphs:

[1] An endoglucanase variant, comprising a substitution at one or more positions corresponding to positions 43, 49, 51, 55, 63, 72, 102, 103, 109, 137, 141, 148, 149, 179, 211, 215, 240, 250, 254, 277, 284, 288, and 320 of the mature polypeptide of SEQ ID NO: 2, wherein the variant has endoglucanase activity.
[2] The variant of paragraph [1], which is a variant of a parent endoglucanase selected from the group consisting of:

(a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, or 40;

(b) a polypeptide encoded by a polynucleotide that hybridizes under low stringency conditions with the full-length complement of the mature polypeptide coding sequence of SEQ ID NO: 1, 3, 5, 7, 9, 11, 13, 15, 17, 19, 21, 23, 25, 27, 29, 31, 33, 35, 37, or 39;

(c) a polypeptide encoded by a polynucleotide having at least 60% identity to the mature polypeptide coding sequence of SEQ ID NO: 1, 3, 5, 7, 9, 11, 13, 15, 17, 19, 21, 23, 25, 27, 29, 31, 33, 35, 37, or 39; and

(d) a fragment of the mature polypeptide of SEQ ID NO: 2, 4, 6, 8, 10, 12, 14, 16, 18, 20, 22, 24, 26, 28, 30, 32, 34, 36, 38, or 40, which has endoglucanase activity.

[3] The variant of paragraph [2], wherein the parent endoglucanase has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99% or 100% sequence identity to the mature polypeptide of SEQ ID NO: 2, 4, 6, 8, 10, 12, 14, 16, 18, 20, 22, 24, 26, 28, 30, 32, 34, 36, 38, or 40.
[4] The variant of paragraph [2] or [3], wherein the parent endoglucanase is encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, 3, 5, 7, 9, 11, 13, 15, 17, 19, 21, 23, 25, 27, 29, 31, 33, 35, 37, or 39 or (ii) the full-length complement of (i).
[5] The variant of any of paragraphs [2]-[4], wherein the parent endoglucanase is encoded by a polynucleotide having at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95%, at least 96%, at least 97%, at least 98%, at least 99%, or 100% sequence identity to the mature polypeptide coding sequence of SEQ ID NO: 1, 3, 5, 7, 9, 11, 13, 15, 17, 19, 21, 23, 25, 27, 29, 31, 33, 35, 37, or 39.
[6] The variant of any of paragraphs [2]-[5], wherein the parent endoglucanase comprises or consists of the mature polypeptide of SEQ ID NO: 2, 4, 6, 8, 10, 12, 14, 16, 18, 20, 22, 24, 26, 28, 30, 32, 34, 36, 38, or 40.
[7] The variant of any of paragraphs [2]-[6], wherein the parent endoglucanase is a fragment of the mature polypeptide of SEQ ID NO: 2, 4, 6, 8, 10, 12, 14, 16, 18, 20, 22, 24, 26, 28, 30, 32, 34, 36, 38, or 40, wherein the fragment has endoglucanase activity.
[8] The variant of any of paragraphs [2]-[7], which has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95% identity, at least 96%, at least 97%, at least 98%, or at least 99%, but less than 100%, sequence identity to the amino acid sequence of the parent endoglucanase.
[9] The variant of any of paragraphs [1]-[8], wherein the variant consists of at least 85%, at least 90%, or at least 95% of the amino acids of a parent.
[10] The variant of any of paragraphs [1]-[9], wherein the number of substitutions is 1-23, e.g., 1, 2, 3, 4, 5, 6, 7, 8, 9, 10, 11, 12, 13, 14, 15, 16, 17, 18, 19, 20, 21, 22, or 23 substitutions.
[11] The variant of any of paragraphs [1]-[10], which comprises a substitution at a position corresponding to position 43.
[12] The variant of paragraph [11], wherein the substitution is with Ser.
[13] The variant of any of paragraphs [1]-[12], which comprises a substitution at a position corresponding to position 49.
[14] The variant of paragraph [13], wherein the substitution is with His.
[15] The variant of any of paragraphs [1]-[14], which comprises a substitution at a position corresponding to position 51.
[16] The variant of paragraph [15], wherein the substitution is with Arg.
[17] The variant of any of paragraphs [1]-[16], which comprises a substitution at a position corresponding to position 55.
[18] The variant of paragraph [17], wherein the substitution is with Lys.
[19] The variant of any of paragraphs [1]-[18], which comprises a substitution at a position corresponding to position 63.
[20] The variant of paragraph [19], wherein the substitution is with Glu, His, or Asn.
[21] The variant of any of paragraphs [1]-[20], which comprises a substitution at a position corresponding to position 72.
[22] The variant of paragraph [21], wherein the substitution is with Arg.
[23] The variant of any of paragraphs [1]-[22], which comprises a substitution at a position corresponding to position 102.
[24] The variant of paragraph [23], wherein the substitution is with Thr.
[25] The variant of any of paragraphs [1]-[24], which comprises a substitution at a position corresponding to position 103.
[26] The variant of paragraph [25], wherein the substitution is with Gly.
[27] The variant of any of paragraphs [1]-[26], which comprises a substitution at a position corresponding to position 109.
[28] The variant of paragraph [27], wherein the substitution is with Lys.
[29] The variant of any of paragraphs [1]-[28], which comprises a substitution at a position corresponding to position 137.
[30] The variant of paragraph [29], wherein the substitution is with Thr.
[31] The variant of any of paragraphs [1]-[30], which comprises a substitution at a position corresponding to position 141.
[32] The variant of paragraph [31], wherein the substitution is with Ala.
[33] The variant of any of paragraphs [1]-[32], which comprises a substitution at a position corresponding to position 148.
[34] The variant of paragraph [33], wherein the substitution is with Thr.
[35] The variant of any of paragraphs [1]-[34], which comprises a substitution at a position corresponding to position 149.
[36] The variant of paragraph [35], wherein the substitution is with His.
[37] The variant of any of paragraphs [1]-[36], which comprises a substitution at a position corresponding to position 179.
[38] The variant of paragraph [37], wherein the substitution is with Thr.
[39] The variant of any of paragraphs [1]-[38], which comprises a substitution at a position corresponding to position 211.
[40] The variant of paragraph [39], wherein the substitution is with His.
[41] The variant of any of paragraphs [1]-[40], which comprises a substitution at a position corresponding to position 215.
[42] The variant of paragraph [41], wherein the substitution is with lie.
[43] The variant of any of paragraphs [1]-[42], which comprises a substitution at a position corresponding to position 240.
[44] The variant of paragraph [43], wherein the substitution is with Val.
[45] The variant of any of paragraphs [1]-[44], which comprises a substitution at a position corresponding to position 250.
[46] The variant of paragraph [45], wherein the substitution is with Gin.
[47] The variant of any of paragraphs [1]-[46], which comprises a substitution at a position corresponding to position 254.
[48] The variant of paragraph [47], wherein the substitution is with Arg.
[49] The variant of any of paragraphs [1]-[48], which comprises a substitution at a position corresponding to position 277.
[50] The variant of paragraph [49], wherein the substitution is with Asn or Val.
[51] The variant of any of paragraphs [1]-[50], which comprises a substitution at a position corresponding to position 284.
[52] The variant of paragraph [51], wherein the substitution is with Arg.
[53] The variant of any of paragraphs [1]-[52], which comprises a substitution at a position corresponding to position 288.
[54] The variant of paragraph [53], wherein the substitution is with Glu, Val, or Tyr.
[55] The variant of any of paragraphs [1]-[54], which comprises a substitution at a position corresponding to position 320.
[56] The variant of paragraph [55], wherein the substitution is with His or Lys.
[57] The variant of any of paragraphs [1]-[56], which comprises a substitution at two positions corresponding to any of positions 43, 49, 51, 55, 63, 72, 102, 103, 109, 137, 141, 148, 149, 179, 211, 215, 240, 250, 254, 277, 284, 288, and 320.
[58] The variant of any of paragraphs [1]-[56], which comprises a substitution at three positions corresponding to any of positions 43, 49, 51, 55, 63, 72, 102, 103, 109, 137, 141, 148, 149, 179, 211, 215, 240, 250, 254, 277, 284, 288, and 320.
[59] The variant of any of paragraphs [1]-[56], which comprises a substitution at four positions corresponding to any of positions 43, 49, 51, 55, 63, 72, 102, 103, 109, 137, 141, 148, 149, 179, 211, 215, 240, 250, 254, 277, 284, 288, and 320.
[60] The variant of any of paragraphs [1]-[56], which comprises a substitution at five positions corresponding to any of positions 43, 49, 51, 55, 63, 72, 102, 103, 109, 137, 141, 148, 149, 179, 211, 215, 240, 250, 254, 277, 284, 288, and 320.
[61] The variant of any of paragraphs [1]-[56], which comprises a substitution at six positions corresponding to any of positions 43, 49, 51, 55, 63, 72, 102, 103, 109, 137, 141, 148, 149, 179, 211, 215, 240, 250, 254, 277, 284, 288, and 320.
[62] The variant of any of paragraphs [1]-[56], which comprises a substitution at seven positions corresponding to any of positions 43, 49, 51, 55, 63, 72, 102, 103, 109, 137, 141, 148, 149, 179, 211, 215, 240, 250, 254, 277, 284, 288, and 320.
[63] The variant of any of paragraphs [1]-[56], which comprises a substitution at eight positions corresponding to any of positions 43, 49, 51, 55, 63, 72, 102, 103, 109, 137, 141, 148, 149, 179, 211, 215, 240, 250, 254, 277, 284, 288, and 320.
[64] The variant of any of paragraphs [1]-[56], which comprises a substitution at nine positions corresponding to any of positions 43, 49, 51, 55, 63, 72, 102, 103, 109, 137, 141, 148, 149, 179, 211, 215, 240, 250, 254, 277, 284, 288, and 320.
[65] The variant of any of paragraphs [1]-[56], which comprises a substitution at ten positions corresponding to any of positions 43, 49, 51, 55, 63, 72, 102, 103, 109, 137, 141, 148, 149, 179, 211, 215, 240, 250, 254, 277, 284, 288, and 320.
[66] The variant of any of paragraphs [1]-[56], which comprises a substitution at eleven positions corresponding to any of positions 43, 49, 51, 55, 63, 72, 102, 103, 109, 137, 141, 148, 149, 179, 211, 215, 240, 250, 254, 277, 284, 288, and 320.
[67] The variant of any of paragraphs [1]-[56], which comprises a substitution at twelve positions corresponding to any of positions 43, 49, 51, 55, 63, 72, 102, 103, 109, 137, 141, 148, 149, 179, 211, 215, 240, 250, 254, 277, 284, 288, and 320.
[68] The variant of any of paragraphs [1]-[56], which comprises a substitution at thirteen positions corresponding to any of positions 43, 49, 51, 55, 63, 72, 102, 103, 109, 137, 141, 148, 149, 179, 211, 215, 240, 250, 254, 277, 284, 288, and 320.
[69] The variant of any of paragraphs [1]-[56], which comprises a substitution at fourteen positions corresponding to any of positions 43, 49, 51, 55, 63, 72, 102, 103, 109, 137, 141, 148, 149, 179, 211, 215, 240, 250, 254, 277, 284, 288, and 320.
[70] The variant of any of paragraphs [1]-[56], which comprises a substitution at fifteen positions corresponding to any of positions 43, 49, 51, 55, 63, 72, 102, 103, 109, 137, 141, 148, 149, 179, 211, 215, 240, 250, 254, 277, 284, 288, and 320.
[71] The variant of any of paragraphs [1]-[56], which comprises a substitution at sixteen positions corresponding to any of positions 43, 49, 51, 55, 63, 72, 102, 103, 109, 137, 141, 148, 149, 179, 211, 215, 240, 250, 254, 277, 284, 288, and 320.
[72] The variant of any of paragraphs [1]-[56], which comprises a substitution at seventeen positions corresponding to any of positions 43, 49, 51, 55, 63, 72, 102, 103, 109, 137, 141, 148, 149, 179, 211, 215, 240, 250, 254, 277, 284, 288, and 320.
[73] The variant of any of paragraphs [1]-[56], which comprises a substitution at eighteen positions corresponding to any of positions 43, 49, 51, 55, 63, 72, 102, 103, 109, 137, 141, 148, 149, 179, 211, 215, 240, 250, 254, 277, 284, 288, and 320.
[74] The variant of any of paragraphs [1]-[56], which comprises a substitution at nineteen positions corresponding to any of positions 43, 49, 51, 55, 63, 72, 102, 103, 109, 137, 141, 148, 149, 179, 211, 215, 240, 250, 254, 277, 284, 288, and 320.
[75] The variant of any of paragraphs [1]-[56], which comprises a substitution at twenty positions corresponding to any of positions 43, 49, 51, 55, 63, 72, 102, 103, 109, 137, 141, 148, 149, 179, 211, 215, 240, 250, 254, 277, 284, 288, and 320.
[76] The variant of any of paragraphs [1]-[56], which comprises a substitution at twenty-one positions corresponding to any of positions 43, 49, 51, 55, 63, 72, 102, 103, 109, 137, 141, 148, 149, 179, 211, 215, 240, 250, 254, 277, 284, 288, and 320.
[77] The variant of any of paragraphs [1]-[56], which comprises a substitution at twenty-two positions corresponding to any of positions 43, 49, 51, 55, 63, 72, 102, 103, 109, 137, 141, 148, 149, 179, 211, 215, 240, 250, 254, 277, 284, 288, and 320.
[78] The variant of any of paragraphs [1]-[56], which comprises a substitution at each position corresponding to positions 43, 49, 51, 55, 63, 72, 102, 103, 109, 137, 141, 148, 149, 179, 211, 215, 240, 250, 254, 277, 284, 288, and 320.
[79] The variant of any of paragraphs [1]-[78], which comprises one or more substitutions selected from the group consisting of G43S; Q49H; L51R; E55K; D63E,H,N; S72R; A102T; D103G; N109K; S137T; T141A; S148T; Q149H; A179T; D211H; S215I; A240V; E250Q; S254R; D277N,V; T284R; D288E,V,Y; and N320H,K.
[80] The variant of paragraph [79], which comprises or consists of the substitutions D63H+E250Q of the mature polypeptide of SEQ ID NO: 2.
[81] The variant of paragraph [79], which comprises or consists of the substitutions T284R+D288V of the mature polypeptide of SEQ ID NO: 2.
[82] The variant of paragraph [79], which comprises or consists of the substitutions L51R+D288Y of the mature polypeptide of SEQ ID NO: 2.
[83] The variant of paragraph [79], which comprises or consists of the substitutions D63H+Q149H+S254R of the mature polypeptide of SEQ ID NO: 2.
[84] The variant of paragraph [79], which comprises or consists of the substitutions D63E+A240V+D277N+N320K of the mature polypeptide of SEQ ID NO: 2.
[85] The variant of paragraph [79], which comprises or consists of the substitutions D63N+A240V+D277N+N320K of the mature polypeptide of SEQ ID NO: 2.
[86] The variant of paragraph [79], which comprises or consists of the substitutions E55K+D63E+D277V+D288E of the mature polypeptide of SEQ ID NO: 2.
[87] The variant of paragraph [79], which comprises or consists of the substitutions A102T+D211H+S215I+N320H of the mature polypeptide of SEQ ID NO: 2.
[88] The variant of paragraph [79], which comprises or consists of the substitutions Q49H+E55K+S148T+D277V of the mature polypeptide of SEQ ID NO: 2.
[89] The variant of paragraph [79], which comprises or consists of the substitutions D63N+D103G+N109K+D277V of the mature polypeptide of SEQ ID NO: 2.
[90] The variant of paragraph [79], which comprises or consists of the substitutions E55K+A179T+S254R+D288E+N320K of the mature polypeptide of SEQ ID NO: 2.
[91] The variant of paragraph [79], which comprises or consists of the substitutions D63H+A240V+D277N+N320K of the mature polypeptide of SEQ ID NO: 2.
[92] The variant of paragraph [79], which comprises or consists of the substitutions G43S+E55K+D363N+S72R+S137T+T141A of the mature polypeptide of SEQ ID NO: 2.
[93] The variant of any of paragraphs [1]-[92], which has an improved property relative to the parent of increased specific performance.
[94] An isolated polynucleotide encoding the variant of any of paragraphs [1]-[93].
[95] A nucleic acid construct comprising the polynucleotide of paragraph [94].
[96] An expression vector comprising the polynucleotide of paragraph [94].
[97] A recombinant host cell comprising the polynucleotide of paragraph [94].
[98] A method of producing an endoglucanase variant, comprising:

(a) cultivating the recombinant host cell of paragraph [97] under conditions suitable for expression of the variant; and optionally

(b) recovering the variant.

[99] A transgenic plant, plant part or plant cell transformed with the polynucleotide of paragraph [94].
[100] A method of producing the variant of any of paragraphs [1]-[93], comprising:

(a) cultivating a transgenic plant or a plant cell comprising a polynucleotide encoding the variant under conditions conducive for production of the variant; and optionally

(b) recovering the variant.

[101] A method for obtaining an endoglucanase variant, comprising introducing into a parent endoglucanase a substitution at one or more positions corresponding to positions 43, 49, 51, 55, 63, 72, 102, 103, 109, 137, 141, 148, 149, 179, 211, 215, 240, 250, 254, 277, 284, 288, and 320 of the mature polypeptide of SEQ ID NO: 2, wherein each alteration is independently a substitution, deletion or insertion and the variant has endoglucanase activity; and recovering the variant.
[102] A whole broth formulation or cell culture composition comprising the variant of any of paragraphs [1]-[93].
[103] An enzyme composition comprising the variant of any of paragraphs [1]-[93].
[104] A process for degrading a cellulosic material, comprising: treating the cellulosic material with an enzyme composition comprising the endoglucanase variant of any of paragraphs [1]-[93].
[105] The process of paragraph [104], wherein the cellulosic material is pretreated.
[106] The process of paragraph [104] or [105], wherein the enzyme composition further comprises one or more enzymes selected from the group consisting of a cellulase, an AA9 polypeptide, a hemicellulase, a cellulose inducible protein (CIP) an esterase, an expansin, a ligninolytic enzyme, an oxidoreductase, a pectinase, a protease, and a swollenin.
[107] The process of paragraph [106], wherein the cellulase is one or more enzymes selected from the group consisting of an endoglucanase, a cellobiohydrolase, and a beta-glucosidase.
[108] The process of paragraph [106], wherein the hemicellulase is one or more enzymes selected from the group consisting of a xylanase, an acetylxylan esterase, a feruloyl esterase, an arabinofuranosidase, a xylosidase, and a glucuronidase.
[109] The process of any of paragraphs [104]-[108], further comprising recovering the degraded cellulosic material.
[110] The process of paragraph [109], wherein the degraded cellulosic material is a sugar.
[111] The process of paragraph [110], wherein the sugar is selected from the group consisting of glucose, xylose, mannose, galactose, and arabinose.
[112] A process for producing a fermentation product, comprising:

(a) saccharifying a cellulosic material with an enzyme composition comprising the endoglucanase variant of any of paragraphs [1]-[93];

(b) fermenting the saccharified cellulosic material with one or more fermenting microorganisms to produce the fermentation product; and

(c) recovering the fermentation product from the fermentation.

[113] The process of paragraph [112], wherein the cellulosic material is pretreated.
[114] The process of paragraph [112] or [113], wherein the enzyme composition further comprises one or more enzymes selected from the group consisting of a cellulase, an AA9 polypeptide, a hemicellulase, a cellulose inducible protein (CIP) an esterase, an expansin, a ligninolytic enzyme, an oxidoreductase, a pectinase, a protease, and a swollenin.
[115] The process of paragraph [114], wherein the cellulase is one or more enzymes selected from the group consisting of an endoglucanase, a cellobiohydrolase, and a beta-glucosidase.
[116] The process of paragraph [114], wherein the hemicellulase is one or more enzymes selected from the group consisting of a xylanase, an acetylxylan esterase, a feruloyl esterase, an arabinofuranosidase, a xylosidase, and a glucuronidase.
[117] The process of any of paragraphs [112]-[116], wherein steps (a) and (b) are performed simultaneously in a simultaneous saccharification and fermentation.
[118] The process of any of paragraphs [112]-[117], wherein the fermentation product is an alcohol, an alkane, a cycloalkane, an alkene, an amino acid, a gas, isoprene, a ketone, an organic acid, or polyketide.
[119] A process of fermenting a cellulosic material, comprising: fermenting the cellulosic material with one or more fermenting microorganisms, wherein the cellulosic material is saccharified with an enzyme composition comprising the endoglucanase variant of any of paragraphs [1]-[93].
[120] The process of paragraph [119], wherein the fermenting of the cellulosic material produces a fermentation product.
[121] The process of paragraph [120], further comprising recovering the fermentation product from the fermentation.
[122] The process of paragraph [120] or [121], wherein the fermentation product is an alcohol, an alkane, a cycloalkane, an alkene, an amino acid, a gas, isoprene, a ketone, an organic acid, or polyketide.
[123] The process of any of paragraphs [119]-[122], wherein the cellulosic material is pretreated before saccharification.
[124] The process of any of paragraphs [119]-[123], wherein the enzyme composition further comprises one or more enzymes selected from the group consisting of a cellulase, an AA9 polypeptide, a hemicellulase, a cellulose inducible protein (CIP) an esterase, an expansin, a ligninolytic enzyme, an oxidoreductase, a pectinase, a protease, and a swollenin.
[125] The process of paragraph [124], wherein the cellulase is one or more enzymes selected from the group consisting of an endoglucanase, a cellobiohydrolase, and a beta-glucosidase.
[126] The process of paragraph [124], wherein the hemicellulase is one or more enzymes selected from the group consisting of a xylanase, an acetylxylan esterase, a feruloyl esterase, an arabinofuranosidase, a xylosidase, and a glucuronidase.

The present invention is further described by the following examples that should not be construed as limiting the scope of the invention.

### Examples

### Media and Solutions

AMG trace metals solution was composed of 14.3 g of ZnSO₄.7H₂O, 2.5 g of CuSO₄.5H₂O, 0.5 g of NiCl₂.6H₂O, 13.8 g of FeSO₄.7H₂O, 8.5 g of MnSO₄.H₂O, 3 g of citric acid, and deionized water to 1 liter.

CBHI medium was composed of 2.5% AVICEL®, 0.5% glucose, and 0.14% (NH₄)₂SO₄ in deionized water.

LB medium was composed of 10 g of tryptone, 5 g of yeast extract, 5 g of sodium chloride, and deionized water to 1 liter.

LB plates were composed of 10 g of tryptone, 5 g of yeast extract, 5 g of sodium chloride, 15 g of Bacto agar, and deionized water to 1 liter.

MDU2BP medium (pH 5.0) was composed of 135 g of maltose, 3 g of MgSO₄.7H₂O, 3 g of NaCl, 6 g of K₂SO₄, 36 g of KH₂PO₄, 21 g of yeast extract, 6 g of urea, 1.5 ml of AMG trace metals solution, and deionized water up to 1 liter.

PDA plates were composed of 39 grams of potato dextrose agar and deionized water to 1 liter.

TAE buffer was composed of 4.82 g of Tris Base, 1.14 ml of Glacial acetic acid, 2 ml of 0.5 M EDTA pH 8.0, and deionized water to 1 liter.

TBE buffer was composed of 10.8 g of Tris Base, 5.5 g boric acid, 4 ml of 0.5 M EDTA pH 8.0, and deionized water to 1 liter.

Digest buffer was composed of 50 mM sodium acetate pH 5.0.

### Example 1: Cloning of Thermoascus aurantiacus GH5 Endoglucanase

Thermoascus aurantiacus NN044936 (T002-5) was grown in 80 ml of CBHI medium in a 500 ml baffled flask at 45° C. with shaking at 165 rpm for 3 days. The mycelia were harvested by centrifugation. Using an RNeasy® Mini Kit (QIAGEN Inc.), total RNA was extracted from 100 mg of the mycelia.

Degenerate primers shown below were designed based on N-terminal amino acid sequencing:

Primer BG025-2:
(SEQ ID NO: 41)
5′-AA(T/C)GA(A/G)TC(T/C/A/G)GG(T/C/A/G)GC(T/C/A/G)
GAG TT-3′
Primer BG025-3:
(SEQ ID NO: 42)
5′-AA(T/C)GA(A/G)AG(T/C)GG(T/C/A/G)GC(T/C/A/G)GAAT
T-3′

A 3′ RACE System (Life Technologies Corp.) was used to synthesize cDNA of the 5′ end of the T. aurantiacus GH5 endoglucanase coding sequence. About 5 μg of total RNA was used as template and the Adapter Primer provided with the 3′ RACE System was used to synthesize the first strand of cDNA.

The cDNA was then amplified by PCR using the degenerate primers above and AUAP provided by the 3′ RACE System. The reactions (50 μl) were composed of 1×PCR buffer, 1.5 mM MgCl₂, 0.2 mM dNTP mix, 0.2 μM primer BG025-2 or primer BG025-3, 0.2 μM AUAP, 2 μl of the cDNA synthesis reaction, and 2.5 units of Taq DNA Polymerase (Life Technologies Corp.). The reaction was performed in a thermocycler programmed for 1 cycle at 94° C. for 3 minutes; and 30 cycles each at 94° C. for 40 seconds, 55° C. for 40 seconds, 72° C. for 1 minute. Then the reaction was incubated at 72° C. for 10 minutes.

The reaction products were isolated by 1.0% low melting point agarose gel electrophoresis using TAE buffer where approximately 1 kb product bands were excised from the gels and purified using a WIZARD® PCR Preps DNA Purification System (Promega Corp.) after incubation at 70° C. for 10 minutes.

The purified fragments were ligated to pGEM®-T Vector (Promega Corp.) according to the manufacturer's protocol. The ligated products were transformed into E. coli JM109 competent cells (Promega Corp.). Transformation colonies were plated onto LB plates supplemented with 100 μg of ampicillin per ml, 0.5 mM isopropyl β-D-1-thiogalactopyranoside (IPTG) per ml, and 80 μg of 5-bromo-4-chloro-3-indolyl-β-D-galactopyranoside (X-Gal) per ml. The plates were incubated overnight at 37° C. Recombinant clones were identified by screening for white colonies which were then inoculated into 3 ml of LB medium supplemented with 100 μg of ampicillin per ml and incubated overnight at 37° C. with shaking at 250 rpm. Minipreps of the DNA was obtained using a Wizard® Plus Minipreps DNA Purification System (Promega Corp.). Recombinant DNA from the clones was sequenced using a DYEnamic™ ET Terminator Cycle Sequencing Kit (GE Healthcare Life Sciences) confirming that the 3′ end of the T. aurantiacus GH5 endoglucanase gene was obtained.

A 5′ RACE System, Version 2 (Life Technologies Corp.) was used to synthesize cDNA of the 5′ end of the T. aurantiacus GH5 endoglucanase coding sequence. For the 5′ RACE reaction, primer BG025-5′-1, which was designed based on the endoglucanase coding sequence obtained from the 3′ RACE reaction, was used for synthesis of the first strand:

Primer BG025-5′-1:
(SEQ ID NO: 43)
5′-AAGATGTACTGGGAAGTG-3′

Second strand synthesis was obtained using two 5′ primers shown below:

Primer BG025-5′-2:
(SEQ ID NO: 44)
5′-TGGTTGAGATTGAGGACTAAG-3′
Primer BG025-5′-4:
(SEQ ID NO: 45)
5′-AGAGCCGGTCATTGAGTTG-3′

The cDNA was amplified by PCR in reactions (50 μl) composed of 1×PCR buffer, 1.5 mM MgCl₂, 0.2 mM dNTP mix, 0.2 μM primer BG025-5′-2′ or primer BG025-5′-4, 0.2 μM Abridged Anchor Primer (Life Technologies Corp.), 5 μl of the cDNA synthesis reaction, and 2.5 units of Taq DNA Polymerase. The reactions were performed in a thermocycler programmed for 1 cycle at 94° C. for 3 minutes; and 30 cycles each at 94° C. for 40 seconds, 53° C. for 40 seconds, and 72° C. for 1 minute. The reactions were then incubated at 72° C. for 10 minutes.

The reaction products were isolated by 1.0% low melting point agarose gel electrophoresis using TAE buffer where bands of approximately 700 bp and 400 bp using primers BG025-5′-2 and BG025-5′-4, respectively, were excised from the gels and purified using a WIZARD® PCR Preps DNA Purification System.

The purified fragments were ligated to pGEM®-T Vector according to the manufacturer's protocol and transformed into E. coli JM109 competent cells similarly as described above. Minipreps of the DNA was obtained using a Wizard® Plus Minipreps DNA Purification System. Recombinant DNA from the clones was sequenced using a DYEnamic™ ET Terminator Cycle Sequencing Kit confirming that the 5′ end of the T. aurantiacus GH5 endoglucanase gene was obtained.

A PCR primer was designed to the 5′ end of the T. aurantiacus GH5 endoglucanase coding sequence to facilitate amplification and subcloning of the full-length cDNA:

Primer BG025-CDS-1:
(SEQ ID NO: 46)
5′-ATGAAGCTCGGCTCTCTCGT-3′

The full-length T. aurantiacus GH5 endoglucanase cDNA was amplified using primer BG025-CDS-1 and AUAP provided by the 3′ RACE System. The reaction (50 μl) was composed of 1×PCR buffer, 1.5 mM MgCl₂, 0.2 mM dNTP mix, 0.2 μM primer BG025-CDS-1, 0.2 μM AUAP, 2 μl of the cDNA synthesis reaction, and 2.5 units of Taq DNA Polymerase. The reaction was performed in a thermocycler programmed for 1 cycle of 94° C. for 3 minutes; and then 30 cycles each at 94° C. for 40 seconds, 55° C. for 40 seconds, and 72° C. for 1 minute. The reaction was then incubated for 72° C. for 10 minutes. The reaction was performed in a thermocycler programmed for 1 cycle at 95° C. for 2 minutes; 30 cycles each at 95° C. for 40 seconds, 58° C. for 40 seconds, and 72° C. for 1.5 minutes; and a final extension at 72° C. for 10 minutes.

The reaction product was isolated by 1.0% low melting point agarose gel electrophoresis using TAE buffer where a single band of approximately 1.2 kb was excised from the gel and purified using a WIZARD® PCR Preps DNA Purification System.

The purified fragment was ligated to pGEM®-T Vector according to the manufacturer's protocol and transformed into E. coli JM109 competent cells similarly as described above. Minipreps of the DNA were obtained using a Wizard® Plus Minipreps DNA Purification System. One positive clone was sequenced using a DYEnamic™ ET Terminator Cycle Sequencing Kit and determined to contain a 1005 bp full-length T. aurantiacus GH5 endoglucanase cDNA. The plasmid was designated pGEM.

### Example 2: Creation of Mutant Libraries

The tailed primers shown below were designed to add PCR priming, T7 promoter, spacer, ribosomal binding site, start codon, and T7 termination sequences to the mature T. aurantiacus GH5 endoglucanase gene sequence. The gene specific portion of the primers is underlined.

Primer A:
(SEQ ID NO: 47)
5′-TTCACGCCCAAAGCATAAACGACGTAATACGACTCACTATAGGGAG
ACCACAAGAAGGAGATATACATATGGCGAAAGTATTCCAATGGT-3′
Primer B:
(SEQ ID NO: 48)
5′-GGGCTAGTTATTGCTCAGCGGTTATTAAAGATACGGAGTCAAAATA
GGAAGT-3′

T. aurantiacus GH5 endoglucanase cDNA (Example 1) was amplified by PCR using the tailed primers in a 50 μl reaction composed of 25 μl of KOD Hot Start Mastermix (Merck KGaA), 0.3 μM Primer A, 0.3 μM Primer B, and 1 ng of cDNA template. The reaction was performed in a thermocycler programmed for 1 cycle at 95° C. for 2 minutes; and 26 cycles each at 95° C. for 20 seconds, 60° C. for 20 seconds, and 68° C. for 45 seconds. The reaction products were treated with 1 μl of Dpn I (20 units/μl; New England Biolabs, Inc.) by incubation at 37° C. for 1 hour to degrade the cDNA template. The treated reaction products were isolated by 0.8% low melting point agarose gel electrophoresis using TAE buffer where approximately 1 kb product bands were excised from the gels and purified using a NucleoSpin® Extract II Kit (Macherey-Nagel) according to the manufacturer's protocol.

The primers shown below were designed to anneal to the ends of the reaction product above.

Primer C:
(SEQ ID NO: 49)
5′-TTCACGCCCAAAGCATAAACGACG-3′
Primer D:
(SEQ ID NO: 50)
5′-GGGGCTAGTTATTGCTCAGCGG-3′

Using a GeneMorph® II Random Mutagenesis Kit (Agilent Technologies), the isolated reaction products above were used as template in 25 μl error-prone PCR reactions composed of 1× Mutazyme II buffer, 0.2 mM dNTP mix, 0.2 μM Primer C, 0.2 μM Primer D, 1.25 units of Mutazyme II DNA polymerase, and 4, 20, or 100 ng of template. The reaction was performed in a thermocycler programmed for 1 cycle at 95° C. for 2 minutes; and 30 cycles each at 95° C. for 30 seconds, 60° C. for 30 seconds, and 72° C. for 1 minute. Then the reaction was incubated at 72° C. for 10 minutes. The error-prone reaction products were isolated by 0.8% low melting point agarose gel electrophoresis using TAE buffer where approximately 1 kb product bands were excised from the gels and purified using a NucleoSpin® Extract II Kit according to the manufacturer's protocol.

### Example 3: Cloning of Thermoascus aurantiacus Endoglucanase Mutants

The primers shown below were designed to introduce suitable tails (lower case) for cloning T. aurantiacus GH5 endoglucanase mutants:

Primer E:
(SEQ ID NO: 51)
5′-tacttccaatccaatgcaGCGAAAGTATTCCAATGGTTCGGT-3′
Primer F:
(SEQ ID NO: 52)
5′-ttatccacttccaatgttattaAAGATACGGAGTCAAAATAGGAA
G-3′

Mutants were amplified by PCR using the tailed primers in a 50 μl reaction composed of 25 μl of KOD Hot Start Mastermix, 0.3 μM Primer E, 0.3 μM Primer F, and approximately 1 pg of error-prone DNA template (see Example 1). The reaction was performed in a thermocycler programmed for 1 cycle at 95° C. for 2 minutes; and 26 cycles each at 95° C. for 20 seconds, 60° C. for 20 seconds, and 68° C. for 45 seconds. The reaction products were isolated by 0.8% low melting point agarose gel electrophoresis using TAE buffer where approximately 1 kb product bands were excised from the gels and purified using a NucleoSpin® Extract II Kit according to the manufacturer's protocol.

Suitable cloning overhangs were generated in a 20 μl reaction composed of 0.2 μmol of isolated reaction products, 1 unit of T4 DNA polymerase (Merck KGaA), 2.5 mM dCTP, 5.0 mM dithiothreitol (DTT), and 1× T4 DNA polymerase buffer (Merck KGaA). The reaction was incubated at 22° C. for 30 minutes and then at 75° C. for 20 minutes. In a 3.4 μl reaction, 0.02 pmol of the resulting products were annealed to 0.014 pmol of a custom expression vector prepared to have complementary overhangs. The custom expression vector contains sequence elements for T7-dependent protein production, stable propagation, and selection in E. coli, and adds an N-terminal 6×His tag followed by a TEV-protease recognition to the T. aurantiacus GH5 endoglucanase variant sequence. The reaction was incubated at 25° C. for 5 minutes followed by the addition of 1 μl of 25 mM EDTA and then incubated at 25° C. for an additional 5 minutes.

### Example 4: Expression of Thermoascus aurantiacus Endoglucanase Variants in E. coli

T. aurantiacus endoglucanase mutants cloned into the custom expression vector of Example 3 were transformed into E. coli SHuffle® competent cells (New England BioLabs). Transformants were plated onto LB plates supplemented with 50 μg of carbenicillin per ml. The plates were incubated overnight at 37° C. Variants were grown in 2 ml LB starter cultures overnight from isolated colonies. Starter culture density was determined by measuring the optical density at 600 nm (OD₆₀₀). Production cultures in MagicMedia™ E. coli expression medium (Life Technologies) were inoculated to OD600 of 0.1 and grown at 37° C., shaking at 250 rpm until they had reached OD600 of 1.0, and then grown at 25° C. with shaking at 250 rpm for a total of 24 hours. Cells were harvested by centrifugation (30 minutes at 3000 rcf) at 4° C., then lysed by sonication using a 400 W Branson Sonifier® (Emerson Electric Co.). Lysed ells were purified on HisTrap FF Crude nickel columns (GE Healthcare Life Sciences) according to the manufacturer's protocol. The 6×His tags were removed from purified proteins with TEV protease (Life Technologies), and then passed through a nickel column as above to remove free tags and TEV protease. After buffer exchange using a XK-10/20 Sephadex G-25 column (GE Healthcare Life Sciences), proteins were quantitated by A₂₈₀, purity and molecular weights were assessed by SDS-PAGE to be >95% and 34 kDa, respectively.

### Example 5: Determination of Specific Performance of Thermoascus aurantiacus Endoglucanase Variants

Purified variants (Example 4) were screened for specific performance using Avicel® PH-101 (FMC Biopolymer) as a substrate as follows. The concentration of purified variants was adjusted to 200 μg/ml in digest buffer (50 mM sodium acetate pH 5.0) and verified by measuring the absorbance at 280 nm with a Spectramax® M5 plate reader (Molecular Devices Corp.). A 2.0% w/v suspension of Avicel® PH-101 was prepared in the digest buffer. Under continuous stirring, 150 μl of the Avicel® suspension was added to each well in a 96-well microplate (Greiner Bio-One). Then, 50 μl of each normalized variant was added to the microplate. Four wells on each 96-well plate contained the wild-type enzyme. Digest plates were heat sealed with heat-sealing foils (Eppendorf EG) and then transferred to incubating shakers where they were agitated at 1000 rpm at 55° C. for 72 hours. Each variant was tested in triplicate, with each replicate performed on a separate digest plate. After digestion, digest plates were centrifuged at 2000 rcf for 5 minutes. A 0.185% w/v solution of p-hydrobenzoic acid hydrazide (pHBAH) was prepared by combining 0.185 g of pHBAH, 0.4 g of NaOH, and 100 μl of water. A total of 225 μl of the pHBAH solution was first added to a 96-well PCR plate (Corning) followed by addition of 25 μl of each digest supernatant and 25 μl of glucose standards at 3, 1, 0.3, 0.1, 0.03, and 0 mM, prepared in digest buffer. Plates were heat sealed as above, and then heated to 95° C. for 5 minutes and cooled to 25° C. for 1 minute in a thermocycler. One hundred μl were then transferred to a half-area microplate (Greiner Bio-One) and the absorbance was measured at 410 nm with a Spectramax® M5 microplate reader. Quantitation of soluble sugars was performed using a linear regression of the glucose standard values.

### Example 6: Multi-Point Dose-Equivalency Measurements of Thermoascus aurantiacus Endoglucanase Variants

The concentration of purified variants was normalized to 1.08 mg/mL in the digest buffer and verified by absorbance at 280 nm using a NanoDrop 1000 spectrophotometer (Thermo Fischer Scientific). Further dilutions were made in the digest buffer to 0.72, 0.54, 0.36, 0.18, 0.90 mg/ml, corresponding to final dose values of 30, 20, 15, 10, 5, 2.5 mg variant per mg of the Avicel® substrate. Multi-point dose-equivalency digests were set up and quantitated as described in Example 5 with the following differences: the first and last row in each plate was not used to avoid plate edge effects; each variant was run as six replicates; wild-type enzyme was run in alternating columns using the same dose-response concentrations for the variants.

The results are shown below in Table I.

TABLE I

| | Dose- | | |
|---|---|---|---|
| | Equivalency | | |
| | Improvement | | Amino Acid |
| ID | (X) | Nucleotide Substitutions | Substitutions |
| AW308 | 2.1X | G130A, G154A, C342T, | E55K, D63N, D277V, |
| | | A797T, C831G | D288E |
| AW325 | 1.9X | G271A, A273T, G598C, | A102T, D211H, |
| | | G611T, A925C | S215I, N320H |
| AW339 | 1.9X | G154C, G715C | D63H, E250Q |
| AW307 | 1.8X | G114T, G130A, G261C, | Q49H, E55K, S148T, |
| | | C303T, T409A, A797T | D277V |
| AW322 | 1.8X | G154A, A275G, T294A, | D63N, D103G, |
| | | A797T | N109K, D277V |
| AW331 | 1.8X | G130A, C234T, G502A, | E55K, A179T, S254R, |
| | | C729A, G762A, C831G, | D288E, N320K |
| | | T927A | |
| AW338 | 1.8X | G154C, G414C, C729A | D63H, Q149H, S254R |
| AW217 | 1.7X | T156G, G474A, C686T, | D63E, A240V, |
| | | G796A, T927A | D277N, N320K |
| AW120 | 1.6X | C501A, C818G, A830T | T284R, D288V |
| AW218 | 1.6X | T119G, C765T, G829T, | L51R, D288Y |
| | | G879A | |
| AW329 | 1.6X | G94A, G130A, G154A, | G43S, E55K, D63N, |
| | | A181C, G261C, T376A, | S72R, S137T, T141A |
| | | A388G | |

### Example 7: Expression of Thermoascus aurantiacus GH5 Endoglucanase Variants in Aspergillus oryzae

Thermoascus aurantiacus wild-type GH5 endoglucanase and variants thereof were expressed in Aspergillus oryzae JaL250. The mature region of each variant was PCR amplified from the respective E. coli constructs and re-fused with the native signal polypeptide into the pAILo2 plasmid (WO 2004/099228).

The following primers were designed for PCR amplification of wild-type T. aurantiacus GH5 signal polypeptide and T. aurantiacus variant mature polypeptide regions for three-way IN-FUSION™ cloning (Clontech Laboratories, Inc.):

1202323: pTH285.F1
(SEQ ID NO: 53)
5′-AATCCTCTATATACACAACTGGATTTACATGAAGCTCGGCTCTCTC
GT-3′
1202324: pTH285.SSJunctF
(SEQ ID NO: 54)
5′-GAAAGCAGGAGACCAAGCGTGCGAAAGTATTCCAATGGTTCG-3′
1202325: pTH285.SSJunctR
(SEQ ID NO: 55)
5′-CGAACCATTGGAATACTTTCGCACGCTTGGTCTCCTGCTTT-3′
1202326: pTH285.R
(SEQ ID NO: 56)
5′-CAGGTGTCAGTCACCTCTAGTTAATTAATCAAAGATACGGAGTCAA
AATAGGAAG-3′

A total of 50 picomoles of each of the primers above were used in an amplification reaction composed of 100 ng of pGEM (Example 1) or plasmid DNA from O12VZG, O12W8X, O12WA5, O12WVF, O12XH1, O12WA3, O12WVB, O12WVD, O12VZK, O12VZJ, O12W8S, or O12WA7; 1× PHUSION® HF Buffer (New England Biolabs, Inc.), 1 μl of a blend of dATP, dTTP, dGTP, and dCTP, each at 10 mM, and 1 unit of PHUSION® DNA polymerase (New England Biolabs, Inc.) in a final volume of 50 μl. The amplification reaction was performed in a thermocycler programmed for 1 cycle at 98° C. for 30 seconds; and 30 cycles each at 98° C. for 10 seconds, 55° C. for 30 seconds, and 72° C. for 1 minute. After the 30 cycles, the reaction was heated for 7 minutes at 72° C. The heat block then went to a 10° C. soak cycle.

The reaction products were isolated by 1.0% agarose gel electrophoresis using TAE buffer where a 140 bp PCR product band containing the GH5 wild-type signal peptide and a 965 bp PCR product band for the wild-type or each variant mature polypeptide were excised from the gel and extracted using a QIAQUICK® Gel Extraction Kit (QIAGEN Inc.).

Plasmid pAILo2 was gapped by digestion with Nco I and Pac I. The digestion was verified by fractionating an aliquot of the digestion by 0.8% agarose gel electrophoresis in TAE buffer where an expected fragment of 5617 bp (gapped) was obtained. The 5617 bp (gapped) fragment was excised from the gel and purified using a QIAQUICK® Gel Extraction Kit.

Multi-fragment PCR cloning was performed according to the method of Zhu et al., 2007, Biotechniques 43(3): 354-359 to construct each of the variant and wild-type endoglucanase A. oryzae expression vectors. The homologous ends of the 140 bp PCR product for the signal polypeptide fragment and the homologous ends of the 965 bp PCR product for the mature polypeptide GH5 wild-type and variant fragments, and plasmid pAILo2, digested with Nco I and Pac I, were joined together using an IN-FUSION™ Advantage PCR Cloning Kit. A total of 50 ng of the 140 bp PCR product, 75 ng of the 965 bp PCR product, and 200 ng of Nco I/Pac I digested pAILo2 were used in a reaction composed of 2 μl of IN-FUSION™ enzyme Premix (Clontech Laboratories, Inc.) in a final volume of 10 μl. The reaction was incubated for 15 minutes at 50° C., and then placed on ice. The reaction volume was increased to 50 μl with 10 mM Tris-0.1 mM EDTA pH 8 (TE) buffer and 3 μl of the reaction was used to transform E. coli XL10-GOLD® Ultracompetent Cells (Stratagene) according to the manufacturer's instructions. Transformants were selected on LB plates supplemented with 100 μg of ampicillin per ml. Plasmid DNA from several of the resulting E. coli transformants was prepared using a BIOROBOT® 9600 (QIAGEN Inc.).

The plasmids were designated O12VZG, O12W8X, 012WA5, O12WVF, O12XH1, O12WA3, O12WVB, O12WVD, O12VZK, O12VZJ, O12W8S, and O12WA7 comprising a polynucleotide encoding full-length wild-type GH5 endoglucanase or variants thereof. The full-length polynucleotide sequences were determined using a 3130xl Genetic Analyzer (Applied Biosystems).

Aspergillus oryzae JaL250 (WO 99/61651) protoplasts prepared according to the method of Christensen et al., 1988, supra, were transformed with 5 μg of plasmid DNA from each of the following E. coli organisms: O12VZG, O12W8X, 012WA5, O12WVF, O12XH1, O12WA3, O12WVB, O12WVD, O12VZK, O12VZJ, O12W8S, and O12WA7. Each transformation yielded about 5-20 transformants. The transformants were spore purified on PDA plates and then grown in 24-well culture plates composed of 1 ml of MDU2BP medium and incubated at 34° C. stationary for 5 days. Broth samples were harvested at day 5 and analyzed by SDS-PAGE using a 8-16% Tris-glycine gel (Bio-Rad Laboratories, Inc.). Once the cultures from each spore purified transformant were confluent and had sporulated, spore stocks were made by applying 5 ml of sterile filtered 0.01% TWEEN® 80 (diluted with glass distilled water) onto the center of each PDA plate and using a sterile spreader to scrape the spores into solution. Spore stocks from the highest producing transformants identified by SDS-PAGE as having darker bands at the predicted molecular weight of approximately 36 kDa were used to inoculate 2 liter shake flasks containing 300 ml of MDU2BP medium. The shake flasks were incubated for 5 days at 34° C. with agitation at 220 rpm. After the incubation, the broths were sterile filtered using a 0.22 μm polyethersulfone membrane (Millipore) for purification. The A. oryzae strains identified from SDS-PAGE analysis of shake flask broths with the darkest band at approximately 36 kDa were designated A. oryzae O32J94, O2254M, O2254N, O32J8V, O32J8U, O32J8Z, O32J8X, O32J8W, O32J92, O32J93, O32J91, and O32J8Y.

| | E. coli | A. oryzae | |
|---|---|---|---|
| Clone ID | Organism ID | Organism ID | Amino Acid substitutions |
| pGEM | O12VZG | O32J94 | Wild - type |
| AW308 | O12W8X | O2254M | E55K, D63N, D277V, D288E |
| AW325 | O12WA5 | O2254N | A102T, D211H, S2151, N320H |
| AW339 | O12WVF | O32J8V | D63H, E250Q |
| AW307 | O12XH1 | O32J8U | Q49H, E55K, S148T, D277V |
| AW322 | O12WA3 | O32J8Z | D63N, D103G, N109K, D277V |
| AW331 | O12WVB | O32J8X | E55K, A179T, S254R, D288E, |
| | | | N320K |
| AW338 | O12WVD | O32J8W | D63H, Q149H, S254R |
| AW217 | O12VZK | O32J92 | D63E, A240V, D277N, N320K |
| AW120 | O12VZJ | O32J93 | T284R, D288V |
| AW218 | O12W8S | O32J91 | L51R, D288Y |
| AW329 | O12WA7 | O32J8Y | G43S, E55K, D63N, S72R, |
| | | | S137T, T141A |

### Example 8: Purification of Thermoascus aurantiacus AW308 Endoglucanase Variant and Wild-Type Endoglucanase

The sterile filtered broths of the Thermoascus aurantiacus AW308 endoglucanase variant and wild-type endoglucanase (Example 7) were concentrated and buffer exchanged into 20 mM Tris pH 8.0 buffer using a tangential flow concentrator with 10 kDa MWCO membrane. The concentrated and buffer exchanged broths (100 ml) were then loaded onto a 200 ml Mono Q column (GE Healthcare Bio-Sciences AB) equilibrated in 20 mM Tris buffer pH 8. Bound protein was eluted using a 0-300 mM NaCl gradient in 20 mM Tris pH 8 buffer. Fractions of 6 ml were collected and analyzed by SDS-PAGE using 8-16% Tris-HCl Stain-Free gels (Bio-Rad Laboratories, Inc.). Fractions with protein of 30 kDa were pooled, and then loaded onto a 320 ml SUPERDEX™ 75 column (GE Healthcare Bio-Sciences AB) equilibrated with 100 mM NaCl-50 mM sodium acetate pH 5. Proteins were eluted with the same buffer (100 mM NaCl in 50 mM sodium acetate pH 5). Fractions of 5 ml were collected. The fractions were analyzed by SDS-PAGE as described above. Fractions with single band at 30 kDa on SDS-PAGE were pooled, and protein content was measured by A₂₈₀.

### Example 9: Preparation of Aspergillus fumigatus GH3 BG-4M

Aspergillus fumigatus GH3 BG-4M variant (F100D+S283G+N456E+F512Y) was prepared recombinantly according to WO 2012/044915. The Aspergillus fumigatus GH3 BG-4M was concentrated and desalted into 50 mM sodium acetate pH 5.0, with 100 mM sodium chloride using a Pall Filtron tangential flow equipped with a Omega 10 kDa membrane (Pall Corporation), and protein was determined using a Microplate BCA™ Protein Assay Kit (Thermo Fischer Scientific) in which bovine serum albumin was used as a protein standard.

### Example 10: Comparison of Thermoascus aurantiacus AW308 Endoglucanase Variant to Wild-Type (Parent) Endoglucanase

The Thermoascus aurantiacus AW308 endoglucanase variant and wild-type endoglucanase were compared in their abilities to hydrolyze microcrystalline cellulose in presence of beta-glucosidase.

The Thermoascus aurantiacus Cel5 AW308 endoglucanase variant and wild-type endoglucanase (E. coli expression) were prepared according to Example 8, and their protein concentrations were based on measurement at A₂₈₀. The Thermoascus aurantiacus AW308 endoglucanase variant and wild-type endoglucanase (A. oryzae expression) were prepared according to Example 9 and purified according to Example 10. The protein concentrations were also based on measurement at A₂₈₀. A. fumigatus GH3 BG-4M was prepared according to Example 9.

A solution of 12 g of AVICEL® PH101 per liter of 50 mM sodium acetate pH 5.0 was prepared. To 150 μl of the AVICEL® solution, 50 μl of an enzyme mixture containing Thermoascus aurantiacus Cel5 endoglucanase variant AW308 or wild-type endoglucanase, at different loadings and A. fumigatus GH3 beta-glucosidase BG-4M were added. A substrate control and enzyme control were included. The final reaction mixture was composed of AVICEL® at 9 g/liter, Thermoascus aurantiacus endoglucanase variant AW308 or wild-type endoglucanase at loadings of 0.5-30 mg/g cellulose, and A. fumigatus GH3 beta-glucosidase BG-4M at a loading of 0.5 mg/g cellulose. The reactions were incubated at 55° C., pH 5.0 for 72 hours. Then samples were filtered through MultiScreen HV 96-well filtration plates (Millipore), and reducing sugars in the filtrate were determined using a p-hydroxybenzoic acid hydrazide (PHBAH, Sigma Chemical Co., Inc.) assay adapted to a 96 well microplate format as described below. A 100 μl aliquot of an appropriately diluted sample was placed in a 96-well PCR plate. 50 μl/well of 1.5% (w/v) PHBAH in 0.5 M NaOH was added to each well and mixed by pipetting up and down. The PCR plate was put in a thermocycler with a rubber lid on top. The plate was heated at 95° C. for 10 minutes, and then cooled to 15° C. for 5 minutes. A 100 μl aliquot from each well was transferred to a flat bottomed 96 well plate and the absorbance at A₄₁₀ₙₘ measured using a SPECTRAMAX® Microplate Reader (Molecular Devices Corp.). Glucose standards (0.1-0.0125 mg/ml diluted with 0.4% sodium hydroxide) were used to prepare a standard curve to convert the obtained A₄₁₀ₙₘ values into glucose equivalents. The resultant equivalents were used to calculate the percentage of cellulose (AVICEL®) conversion for each reaction. The degree of AVICEL® conversion to reducing sugar (conversion, %) was calculated using the following equation:

Conversion (%)=RS_{(mg/ml)}*100*162/(Avicel_{(mg/ml)}*180)=RS_{(mg/ml)}*100/(Avicel_{(mg/ml)}*1.111)

In this equation, RS is the concentration of reducing sugar in solution measured in glucose equivalents (mg/ml), and the factor 1.111 reflects the weight gain in converting cellulose to glucose.

TABLE 1
Avicel hydrolysis with presence of BG.

| Ta GH5 | | | | | | | | |
|---|---|---|---|---|---|---|---|---|
| Loading, | Avg Conversion, % | | | | Std dev, Conversion, % | | | |
| mg/g | AW308, Ec | WT, Ec | AW308, Ao | WT, Ao | AW308, Ec | WT, Ec | AW308, Ao | WT, Ao |
| cellulose | expressed | expressed | expressed | expressed | expressed | expressed | expressed | expressed |
| 0.5 | 0.85% | 1.09% | 0.96% | 1.13% | 0.03% | 0.02% | 0.05% | 0.01% |
| 1 | 1.13% | 1.29% | 1.27% | 1.36% | 0.02% | 0.01% | 0.04% | 0.03% |
| 2.5 | 1.53% | 1.53% | 1.60% | 1.61% | 0.02% | 0.03% | 0.04% | 0.04% |
| 5.0 | 1.81% | 1.72% | 1.86% | 1.78% | 0.05% | 0.01% | 0.06% | 0.00% |
| 10.0 | 1.93% | 1.75% | 1.97% | 1.79% | 0.06% | 0.02% | 0.04% | 0.01% |
| 15.0 | 1.89% | 1.75% | 1.94% | 1.80% | 0.05% | 0.00% | 0.03% | 0.02% |
| 20.0 | 1.96% | 1.77% | 1.98% | 1.84% | 0.07% | 0.01% | 0.04% | 0.03% |
| 30.0 | 1.99% | 1.87% | 2.10% | 1.90% | 0.06% | 0.02% | 0.07% | 0.02% |

Variant AW308 (both E. coli expressed and A. oryzae expressed) showed better performance on AVICEL® than the corresponding wild-type endoglucanase at loadings of 5-30 mg/g cellulose.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An endoglucanase variant, comprising a substitution at one or more positions corresponding to positions 43, 49, 51, 55, 63, 72, 102, 103, 109, 137, 141, 148, 149, 179, 211, 215, 240, 250, 254, 277, 284, 288, and 320 of the mature polypeptide of SEQ ID NO: 2, wherein the variant has endoglucanase activity.

2. The variant of claim 1, which is a variant of a parent endoglucanase selected from the group consisting of:
(a) a polypeptide having at least 60% sequence identity to the mature polypeptide of SEQ ID NO: 2, 4, 6, 8, 10, 12, 14, 16, 18, 20, 22, 24, 26, 28, 30, 32, 34, 36, 38, or 40;
(b) a polypeptide encoded by a polynucleotide that hybridizes under low stringency conditions with the full-length complement of the mature polypeptide coding sequence of SEQ ID NO: 1, 3, 5, 7, 9, 11, 13, 15, 17, 19, 21, 23, 25, 27, 29, 31, 33, 35, 37, or 39;
(c) a polypeptide encoded by a polynucleotide having at least 60% identity to the mature polypeptide coding sequence of SEQ ID NO: 1, 3, 5, 7, 9, 11, 13, 15, 17, 19, 21, 23, 25, 27, 29, 31, 33, 35, 37, or 39; and
(d) a fragment of the mature polypeptide of SEQ ID NO: 2, 4, 6, 8, 10, 12, 14, 16, 18, 20, 22, 24, 26, 28, 30, 32, 34, 36, 38, or 40, which has endoglucanase activity.

3. The variant of claim 2, wherein the parent endoglucanase comprises or consists of the mature polypeptide of SEQ ID NO: 2, 4, 6, 8, 10, 12, 14, 16, 18, 20, 22, 24, 26, 28, 30, 32, 34, 36, 38, or 40.

4. The variant of claim 2, which has at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 95% identity, at least 96%, at least 97%, at least 98%, or at least 99%, but less than 100%, sequence identity to the amino acid sequence of the parent endoglucanase.

5. The variant of claim 1, which comprises one or more substitutions selected from the group consisting of G43S; Q49H; L51R; E55K; D63E,H,N; S72R; A102T; D103G; N109K; S137T; T141A; S148T; Q149H; A179T; D211H; S215I; A240V; E250Q; S254R; D277N,V; T284R; D288E,V,Y; and N320H,K.

6. The variant of claim 5, which comprises or consists of the substitutions D63H+E250Q of the mature polypeptide of SEQ ID NO: 2.

7. The variant of claim 5, which comprises or consists of the substitutions T284R+D288V of the mature polypeptide of SEQ ID NO: 2.

8. The variant of claim 5, which comprises or consists of the substitutions L51R+D288Y of the mature polypeptide of SEQ ID NO: 2.

9. The variant of claim 5, which comprises or consists of the substitutions D63H+Q149H+S254R of the mature polypeptide of SEQ ID NO: 2.

10. The variant of claim 5, which comprises or consists of the substitutions D63E+A240V+D277N+N320K of the mature polypeptide of SEQ ID NO: 2.

11. The variant of claim 5, which comprises or consists of the substitutions D63N+A240V+D277N+N320K of the mature polypeptide of SEQ ID NO: 2.

12. The variant of claim 5, which comprises or consists of the substitutions E55K+D63E+D277V+D288E of the mature polypeptide of SEQ ID NO: 2.

13. The variant of claim 5, which comprises or consists of the substitutions A102T+D211H+S215I+N320H of the mature polypeptide of SEQ ID NO: 2.

14. The variant of claim 5, which comprises or consists of the substitutions Q49H+E55K+S148T+D277V of the mature polypeptide of SEQ ID NO: 2.

15. The variant of claim 5, which comprises or consists of the substitutions D63N+D103G+N109K+D277V of the mature polypeptide of SEQ ID NO: 2.

16. The variant of claim 5, which comprises or consists of the substitutions E55K+A179T+S254R+D288E+N320K of the mature polypeptide of SEQ ID NO: 2.

17. The variant of claim 5, which comprises or consists of the substitutions D63H+A240V+D277N+N320K of the mature polypeptide of SEQ ID NO: 2.

18. The variant of claim 5, which comprises or consists of the substitutions G43S+E55K+D363N+S72R+S137T+T141A of the mature polypeptide of SEQ ID NO: 2.

19. An isolated polynucleotide encoding the variant of claim 1.

20. A recombinant host cell comprising the polynucleotide of claim 19.

21. A method of producing an endoglucanase variant, comprising:
(a) cultivating the recombinant host cell of claim 20 under conditions suitable for expression of the variant; and optionally
(b) recovering the variant.

22. A transgenic plant, plant part or plant cell transformed with the polynucleotide of claim 19.

23. A method of producing the variant of claim 1, comprising:
(a) cultivating a transgenic plant or a plant cell comprising a polynucleotide encoding the variant under conditions conducive for production of the variant; and optionally
(b) recovering the variant.

24. A method for obtaining an endoglucanase variant, comprising introducing into a parent endoglucanase a substitution at one or more positions corresponding to positions 43, 49, 51, 55, 63, 72, 102, 103, 109, 137, 141, 148, 149, 179, 211, 215, 240, 250, 254, 277, 284, 288, and 320 of the mature polypeptide of SEQ ID NO: 2, wherein each alteration is independently a substitution, deletion or insertion and the variant has endoglucanase activity; and recovering the variant.

25. A process for degrading a cellulosic material, comprising: treating the cellulosic material with an enzyme composition comprising the endoglucanase variant of claim 1; and recovering the degraded cellulosic material.

26. A process for producing a fermentation product, comprising:
(a) saccharifying a cellulosic material with an enzyme composition comprising the endoglucanase variant of claim 1;
(b) fermenting the saccharified cellulosic material with one or more fermenting microorganisms to produce the fermentation product; and
(c) recovering the fermentation product from the fermentation.


SEQUENCE LISTING (56 sequences)

SEQ ID NO: 1 - DNA, 1008 bp, Thermoascus aurantiacus
atgaagctcggctctctcgtgctcgctctcagcgcagctaggcttacactgtcggcccctctcgcagacagaaagcaggagaccaagcgtgcgaaagtattccaatggttcggttcgaacgagtccggtgctgaattcggaagccagaaccttccaggagtcgagggaaaggattatatatggcctgatcccaacaccattgacacattgatcagcaaggggatgaacatctttcgtgtcccctttatgatggagagattggttcccaactcaatgaccggctctccggatccgaactacctggcagatctcatagcgactgtaaatgcaatcacccagaaaggtgcctacgccgtcgtcgatcctcataactacggcagatactacaattctataatctcgagcccttccgatttccagaccttctggaaaacggtcgcctcacagtttgcttcgaatccactggtcatcttcgacactaataacgaataccacgatatggaccagaccttagtcctcaatctcaaccaggccgctatcgacggcatccgttccgccggagccacttcccagtacatctttgtcgagggcaattcgtggaccggggcatggacctggacgaacgtgaacgataacatgaaaagcctgaccgacccatctgacaagatcatatacgagatgcaccagtacctggactctgacggatccgggacatcagcgacctgcgtatcttcgaccatcggtcaagagcgaatcaccagcgcaacgcagtggctcagggccaacgggaagaagggcatcatcggcgagtttgcgggcggagccaacgacgtctgcgagacggccatcacgggcatgctggactacatggcccagaacacagacgtctggactggcgccatctggtgggcggccgggccgtggtggggagactacatattctccatggagccggacaatggcatcgcgtatcagcagatacttcctattttgactccgtatctttga

SEQ ID NO: 2 - protein, 335 aa, Thermoascus aurantiacus
MKLGSLVLALSAARLTLSAPLADRKQETKRAKVFQWFGSNESGAEFGSQNLPGVEGKDYIWPDPNTIDTLISKGMNIFRVPFMMERLVPNSMTGSPDPNYLADLIATVNAITQKGAYAVVDPHNYGRYYNSIISSPSDFQTFWKTVASQFASNPLVIFDTNNEYHDMDQTLVLNLNQAAIDGIRSAGATSQYIFVEGNSWTGAWTWTNVNDNMKSLTDPSDKIIYEMHQYLDSDGSGTSATCVSSTIGQERITSATQWLRANGKKGIIGEFAGGANDVCETAITGMLDYMAQNTDVWTGAIWWAAGPWWGDYIFSMEPDNGIAYQQILPILTPYL

SEQ ID NO: 3 - DNA, 1005 bp, Penicillium emersonii
atgaagttcagcagagtcgtctgcggtctggcagccgcagggggcgccctcgccgcgccagccaaggagaagagtatcaagaagcgggcgtctccgtttcaatggttcggatccaatgagtccggcgcagagtttggacaaaacaacattcctggcgtggagggcaccgactacaccttccccaacaccaccgccatccaggtcctgatcgaccagggcatgaacatcttccgcgtgcccttcctgatggagcgcatggtgcccaatcagatgaccggccccgtggattcggcgtatctgcagggctacagccaggtgatcaactacattaccagccacggggcgtcggcggtgattgacccacataactatgggagatactacaacaatatcatctcctcgccgtcggacttccagactttctggaacactattgcgtccaactttgcggacaatgacggcgtgattttcgacacgaacaacgaataccacgacatggacgaaagtctcgtcgtgcagctcaaccaggccgccatcgacggcatccgcgcggcgggcgccacgtcgcagtacatcttcgtggaaggaaactcgtggacgggggcctggacgtggacgcaggtaaacgacgcgatggcgaacctgacagatccccagaacaagatcgtctacgagatgcaccagtacctcgactcggacgggtcggggacgtcggaccagtgtgtcaacgcgaccatcgggcaggaccgcgtcgcgtccgccacggcctggctgaagcagaacggcaagaaggcgattctgggcgagttcgcgggcggtgcgaacagcgtgtgcgagtccgccgtcacgggcctgctcgaccatctggccgataatacggatgtctggacgggggctatctggtgggctgctgggccgtggtgggcagactacatcttctccatggagccgccgagtgggattgcgtatgagcaggttctgccgttgctgcagccatatctttga

SEQ ID NO: 4 - protein, 334 aa, Penicillium emersonii
MKFSRVVCGLAAAGGALAAPAKEKSIKKRASPFQWFGSNESGAEFGQNNIPGVEGTDYTFPNTTAIQVLIDQGMNIFRVPFLMERMVPNQMTGPVDSAYLQGYSQVINYITSHGASAVIDPHNYGRYYNNIISSPSDFQTFWNTIASNFADNDGVIFDTNNEYHDMDESLVVQLNQAAIDGIRAAGATSQYIFVEGNSWTGAWTWTQVNDAMANLTDPQNKIVYEMHQYLDSDGSGTSDQCVNATIGQDRVASATAWLKQNGKKAILGEFAGGANSVCESAVTGLLDHLADNTDVWTGAIWWAAGPWWADYIFSMEPPSGIAYEQVLPLLQPYL

SEQ ID NO: 5 - DNA, 1260 bp, Chaetomium virescens
atgccttcagtcgtcctttctcttgctttcttggcgggcagtgcccttgcccagcagagtgtctggggacaatgcggtggaataggctggattgggccaacaaactgcgcggctgggagctgctgcgctacccagaacgcctggtatgcacaatgtacgcccggggtggcatgcggtggtggtggtagtggtacgactttgacaaccatgacaacccggaccacgactgttcaggccacgactacgaccgttacatcgacccccaccagcaacggcaaggttcgcttcgccggcgtcaacattgcaggcttcgactttgggtgcgggaccgatggcaactgtaataccgcaaaggtgtggccgccagtgaagaactaccccccagactacaaccaccccgacggcgccggtcagatgcagcacttctacaaggatgaccacatgaacatcttccgcctgcccgtgggttggcaatacctggtcaacaacaacctcggcggcaacttggacagcaccaacttcggctactacgaccagctggtccaggcctgcctcaacacgggcgcatactgcatcatcgacatccacaactacgcgcggtggaatggggccattatcggccagggcgggcccacaaacgagcagttcgtcagcatatggaagcagctggcgagcaagtacgccagccagtcgcgcatatggttcggcatcatgaacgagccccacgacatacccaatatcaacacctgggcggccacggttcaggccgtcgtcacagccatccgcaatgccggcgccaccagccagttcatctcgctgcccggcaacgggtggcagtccgcgggcaccttcatcagcgacggaagcgctgcggctctgtctaccgtcaggaacccggatggatcgacgacgaacctcatttttgatgtgcacaagtacctcgattctgacaactcggggacgcataccgagtgtgtcaagaataatatcgacgacgcgtttgcgccgcttgccgcctggctcaggcaaaataacagacaggcgatcctcactgagacgggaggcggcaacacggcttcatgccagacttatgtgtgccagcagattgcgtacctgaatcaaaactcggacgtctatctcggctatgtcggctgggctgctggttcgttcgacaatacgtacgagctggtcgagacgccgacctggaatggtaactcgtttagtgatacggcccttgtcaggtcttgccttgctcgttaa

SEQ ID NO: 6 - protein, 419 aa, Chaetomium virescens
MPSVVLSLAFLAGSALAQQSVWGQCGGIGWIGPTNCAAGSCCATQNAWYAQCTPGVACGGGGSGTTLTTMTTRTTTVQATTTTVTSTPTSNGKVRFAGVNIAGFDFGCGTDGNCNTAKVWPPVKNYPPDYNHPDGAGQMQHFYKDDHMNIFRLPVGWQYLVNNNLGGNLDSTNFGYYDQLVQACLNTGAYCIIDIHNYARWNGAIIGQGGPTNEQFVSIWKQLASKYASQSRIWFGIMNEPHDIPNINTWAATVQAVVTAIRNAGATSQFISLPGNGWQSAGTFISDGSAAALSTVRNPDGSTTNLIFDVHKYLDSDNSGTHTECVKNNIDDAFAPLAAWLRQNNRQAILTETGGGNTASCQTYVCQQIAYLNQNSDVYLGYVGWAAGSFDNTYELVETPTWNGNSFSDTALVRSCLAR

SEQ ID NO: 7 - DNA, 1191 bp, Talaromyces pinophilus
atgaggtctacatcaacatttgtagctagtgctatactagcagtcgcttccattcaagcccagcagactggatatggccagtgcggtggtgagaactggactggtgccacgacctgcgtgtctggttggacatgtacctatcttaacgattggtactctcaatgtctaccagcttccagcactttgactaccacgacatcttcgaagacctctactactgctacaacgacttcaaagactacaacctcatcgacgagttcaccgacgagtaccggaaaattgaaatggttcggtgtagatgaatcatgtgccgagttcggaactgcaatgccaggcacttggggtgtcgacttcaccttcgccaatacagcaaccattggtgaattcatcagtcagggtttcaatattttccgtatcccctttgccatggaacgaatggtacaagggtcaattgacgctgctttaaataccgcatatttgaccaactactcagttgccgtcaactatattacctcgaatggagcatacgccgtgattgatcctcacaattatggaaggtacaatggcagcatcatcaccgatactactgctttccagactttctggtctaacttggccactgctttcaagagcaattccaaagttatctttgacacaaacaacgagtatcacgacatggatgaaaccctggtttttaacctgaaccaagcagcaattgacggtattcgtggcgccggagccacaacgcaatatatctttgccgaaggtaatagctggactggagcatggacctggaacacgaccaatgattcactcaaagatctaagtgatcctgagaacctacttgtctacgaaatgcaccaataccttgactctgatggatctggcacaaattctgcctgcgtctcctcaacaattggtgtcgagcgtgtagaaggtgccactgcttggttacaggcgaacaaaaagctcggtgttctaggagagtatgccggtggccccaactcggtctgtcaagccgccgtaacaggaatgctggaccatctagtcgccaacaacgacgtctggctaggagccgtgtggtgggcggctggtccatggtggccttcttcgacttgggcaagcattgagccaccgagtggacaggcttatgtctattatgatgaaatcctgcaggcctacaccccttag

SEQ ID NO: 8 - protein, 396 aa, Talaromyces pinophilus
MRSTSTFVASAILAVASIQAQQTGYGQCGGENWTGATTCVSGWTCTYLNDWYSQCLPASSTLTTTTSSKTSTTATTTSKTTTSSTSSPTSTGKLKWFGVDESCAEFGTAMPGTWGVDFTFANTATIGEFISQGFNIFRIPFAMERMVQGSIDAALNTAYLTNYSVAVNYITSNGAYAVIDPHNYGRYNGSIITDTTAFQTFWSNLATAFKSNSKVIFDTNNEYHDMDETLVFNLNQAAIDGIRGAGATTQYIFAEGNSWTGAWTWNTTNDSLKDLSDPENLLVYEMHQYLDSDGSGTNSACVSSTIGVERVEGATAWLQANKKLGVLGEYAGGPNSVCQAAVTGMLDHLVANNDVWLGAVWWAAGPWWPSSTWASIEPPSGQAYVYYDEILQAYTP

SEQ ID NO: 9 - DNA, 1230 bp, Talaromyces leycettanus
atgaagttttccaacgtgattcttgcggccagcgcgtcgagcctggtgctcgctgctcccaagagcaagaccaagcgcacatctgccttccaatggttcggtgccaacgaatctggcgctgaattcggcaaccagaacatccccggcactcttggaacggattacacctggcccgacacttcgaccatccagactctgcgcaatgcgggcatgaacatcttccgtgtccccttcttgatggagagactggttcccaaccagatgaccggtagcccggatcccacctatttggctgatttgaaatcgactgtcaacttcatcaccggtactggtgcttatgccgttgtcgaccctcacaactatggccgatactacaacaatatcatcacctcgacctctgactttgctgccttctggacgacagttgcctcgcagtttgcctccaatcccagggtcatcttcgacaccaacaacgagtacaacaacatggaccaaacgctcgttctcaacctgaaccaggccgccatcaacgccatccgcgccgccggcgccacctcgcagtacattttcgccgagggcaactcctggactggcgcgtggacctggaccagcgtcaacgacaacatgaagcagctgactgatccttccaacaagctcgtctacgagatgcaccagtacctcgactccgacggctcgggcacgtcggaccagtgcgtcaactcgaccattggctacgaccgcatcgtgagcgccacgcaatggctccaggcgaacggcaaggtcgccttcctgggcgaatttgccggcggctccaacagcgtctgcgaggctgccgtgacgggcatgctcgactacatggagcagaacagcgacgtctggctcggggcggaatggtgggccgctggtccctggtggggcaactacatctacagcatggagcccccgagtggcattgcgtatcagaactacctgtcgatcttggagccttacttccccggcggctcttactctggtggcactggatcaggatctggttcgacgacgacaacggcgacgacgacgacgacgaaagtgccgcccacttcgaccacctccagcgcttccagcaccggtactggcgttgctcagcactggggccagtgcggtggccagggatggaccggtccgaccacctgcgttagcccttacacctgccaggagctgaacccctattactaccagtgcctgtag

SEQ ID NO: 10 - protein, 409 aa, Talaromyces leycettanus
MKFSNVILAASASSLVLAAPKSKTKRTSAFQWFGANESGAEFGNQNIPGTLGTDYTWPDTSTIQTLRNAGMNIFRVPFLMERLVPNQMTGSPDPTYLADLKSTVNFITGTGAYAVVDPHNYGRYYNNIITSTSDFAAFWTTVASQFASNPRVIFDTNNEYNNMDQTLVLNLNQAAINAIRAAGATSQYIFAEGNSWTGAWTWTSVNDNMKQLTDPSNKLVYEMHQYLDSDGSGTSDQCVNSTIGYDRIVSATQWLQANGKVAFLGEFAGGSNSVCEAAVTGMLDYMEQNSDVWLGAEWWAAGPWWGNYIYSMEPPSGIAYQNYLSILEPYFPGGSYSGGTGSGSGSTTTTATTTTTKVPPTSTTSSASSTGTGVAQHWGQCGGQGWTGPTTCVSPYTCQELNPYYYQCL

SEQ ID NO: 11 - DNA, 1002 bp, Talaromyces pinophilus
atgaagaccagcattatttctatcgttctgtctacggcaggactcactttaggggccccctcaaaggacaccaagaaacgtgcttcaagtttcgaatggttcggttcaaatgagtccggagcagaatttggaagtgggaatatcccaggtgtggagggcaccgactacacctttcccaatacaacagcgattcaaatactcatcgacgccggtatgaacatcttccgcgttccattcctaatggagcgaatgatcccgactgagatgactggatctcttaatacggcttattttgaggggtacagcgaggtcattaactacatcaccggtcaaggagcacatgcagtggttgaccctcacaactttggacgatattatggaacccctatctcatcaacatccgacttccagactttctggtccacgcttgcctcccaattcaaatcaaatgacaaggtcatttttgacacaaacaacgaataccacgacatggatgaatccgtcgtcgtagccctaaaccaagcagcaatcgacggcatccgcgatgccggggccacaacacaatacatcttcgtcgaaggcaactcttacactggtgcctggacatggacaacatacaacacggccatggtgaacctcaccgatgcatctgatctaatcgtctacgaaatgcatcaatacctcgactctgacgggtctggtacatcagaccaatgcgtgagcagcacaatcggccaggaacgtgttgtagatgctacaacttggttgcaaaccaacggaaagcgaggcatccttggcgaattcgcgggtggcgcaaacagtgtttgcgaagaggccgtgaaggggatgctgaattatctggagcagaattccgacgtctggctcggagcgagctggtggagtgcgggcccatggtggggtgactacattttctcaatggaaccacctagcggcactgcgtatgtgaattatctgtcgattctggagagttatttcccttga

SEQ ID NO: 12 - protein, 333 aa, Talaromyces pinophilus
MKTSIISIVLSTAGLTLGAPSKDTKKRASSFEWFGSNESGAEFGSGNIPGVEGTDYTFPNTTAIQILIDAGMNIFRVPFLMERMIPTEMTGSLNTAYFEGYSEVINYITGQGAHAVVDPHNFGRYYGTPISSTSDFQTFWSTLASQFKSNDKVIFDTNNEYHDMDESVVVALNQAAIDGIRDAGATTQYIFVEGNSYTGAWTWTTYNTAMVNLTDASDLIVYEMHQYLDSDGSGTSDQCVSSTIGQERVVDATTWLQTNGKRGILGEFAGGANSVCEEAVKGMLNYLEQNSDVWLGASWWSAGPWWGDYIFSMEPPSGTAYVNYLSILESYFP

SEQ ID NO: 13 - DNA, 1257 bp, Trichoderma reesei
atgaacaagtccgtggctccattgctgcttgcagcgtccatactatatggcggcgccgtcgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactagcagtggtaactcatggacggacacatccttggtcagctcgtgtctcgcaagaaagtag

SEQ ID NO: 14 - protein, 418 aa, Trichoderma reesei
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTSSGNSWTDTSLVSSCLARK

SEQ ID NO: 15 - DNA, 1008 bp, Talaromyces emersonii
atgaagttcagcagggtcgtgtgcggtctgacggccgcagggggcgccctcgccgctccagtcaaggagaagggcatcaagaagcgggcgtctccgtttcaatggttcggatccaacgagtctggcgcagagtttgggaacaacaacatccctggcgtggagggcaccgactacaccttccccaacacgagcgccatccagatcctcatcgaccagggcatgaacatcttccgcgtgccgttcctgatggagcgcatggtgcccaaccagatgacggggccggtggattcggcgtatttccagggctacagccaggttatcaactacattaccagccatggcgcgtcggcagtgattgacccgcataacttcgggcgatactacaacaatatcatctcctcgccgtctgacttccagactttctggcacactattgcgtccaactttgcggataatgacaatgtcattttcgacacgaacaacgaataccacgacatggacgaaagccttgtcgtccagctcaaccaggccgccatcgacggcatccgcgccgcgggcgccacatcacagtacatcttcgtcgagggcaactcgtggaccggggcctggacatggacgcaggtcaacgacgcgatggcgaacctgacggacccgcagaacaagatcgtgtacgagatgcaccagtacctggactcggacgggtcgggcacgtcggaccagtgcgtcaactcgaccatcgggcaggaccgcgtcgagtcggcgacggcctggctgaagcagaacggcaagaaggcgatcctgggcgagtacgctggcggcgccaacagcgtgtgcgagacggccgtcaccggcatgctcgactatctcgccaacaatactgatgtctggaccggtgctatctggtgggcggctgggccgtggtggggagactatatcttctccatggagccgcctagtgggattgcgtatgagcaggttctgccgttgctgaagccgtacctcgaatga

SEQ ID NO: 16 - protein, 335 aa, Talaromyces emersonii
MKFSRVVCGLTAAGGALAAPVKEKGIKKRASPFQWFGSNESGAEFGNNNIPGVEGTDYTFPNTSAIQILIDQGMNIFRVPFLMERMVPNQMTGPVDSAYFQGYSQVINYITSHGASAVIDPHNFGRYYNNIISSPSDFQTFWHTIASNFADNDNVIFDTNNEYHDMDESLVVQLNQAAIDGIRAAGATSQYIFVEGNSWTGAWTWTQVNDAMANLTDPQNKIVYEMHQYLDSDGSGTSDQCVNSTIGQDRVESATAWLKQNGKKAILGEYAGGANSVCETAVTGMLDYLANNTDVWTGAIWWAAGPWWGDYIFSMEPPSGIAYEQVLPLLKPYLE

SEQ ID NO: 17 - DNA, 999 bp, Penicillium spinulosum
atgaagtttcagagcactctgcttcttgccgccgcggctggttccgcgttggctgtgcctcatggtcctggacataagaagagggcgtctgtgtttgaatggttcggatcgaatgagtctggtgctgagtttgggaccaatattcctggggtttggggaaccgactacatcttccccgacccctcggccatctctacgttgattgacaaggggatgaacttcttccgcgtccagttcatgatggagaggttgctgcccgactcgatgactggctcatatgatgaggagtatctggccaatttgacgacagtgataaaagcggtaacggacggaggcgctcatgcccttgttgaccctcataactatggcagatacaacggcgagatcatctccagcacgtctgatttccagaccttctgggaaaatctggccggccagtacaaagataacgacctggtcatgtttgacactaacaacgaatatcacgacatggatcaggatctcgtgctgaacctcaaccaagcagccattaacggcatccgcgccgcaggtgcgaccagccagtacatcttcgtcgaaggcaactcctggaccggagcctggacgtgggtcgacgtcaatgacaacatgaagaatttgaccgaccccgaagacaagatcgtctatgaaatgcatcagtacctagactccgacggttccggcacctcggagacctgcgtgtccgagaccatcggaaaagagcgggtcactgaagctacacagtggctgaaggacaataagaaggtcggcttcataggcgaatatgccgggggttccaatgatgtatgtcggagtgccgtgtcggggatgctggagtacatggcgaataacaccgacgtatggaagggtgcgtcgtggtgggcagccgggccatggtggggagactacattttcagcctggagcccccagatggaactgcgtacacgggtatgctggatatcctggaggcgtatctttga

SEQ ID NO: 18 - protein, 332 aa, Penicillium spinulosum
MKFQSTLLLAAAAGSALAVPHGPGHKKRASVFEWFGSNESGAEFGTNIPGVWGTDYIFPDPSAISTLIDKGMNFFRVQFMMERLLPDSMTGSYDEEYLANLTTVIKAVTDGGAHALVDPHNYGRYNGEIISSTSDFQTFWENLAGQYKDNDLVMFDTNNEYHDMDQDLVLNLNQAAINGIRAAGATSQYIFVEGNSWTGAWTWVDVNDNMKNLTDPEDKIVYEMHQYLDSDGSGTSETCVSETIGKERVTEATQWLKDNKKVGFIGEYAGGSNDVCRSAVSGMLEYMANNTDVWKGASWWAAGPWWGDYIFSLEPPDGTAYTGMLDILEAYL

SEQ ID NO: 19 - DNA, 990 bp, Aspergillus fumigatus
atgaaattcggtagcattgtgctcattgctgctgcggcaggcttcgcggtggctgctcctgcaaagagagcttcggtatttcaatggttcggaagcaatgagtctggagcagagtttggcgaaaataccattcctggctcttatgggaaagaattcatcttcccggacccttctacaatcagcacattgatcgggaagggcatgaacatcttccggattcaattcctcatggagagactggtgccaagctctatgacaggctcctataacgaggagtaccttgccaatctgacatcggttgtggacgctgtcaccaaggcaggatcttatgctattttggacccacacaactttggcagatacaatggtcagattatctccagcaccgacgacttcaagaccttctggcagaatctggctggaaagttcaagtccaacaatctcgtcatctttgatactaacaatgagtatcacgacatggaccagacactggtactgaacctcaaccaggccgctatcaacggtatccgcgctgcaggagccacctcgcaatacatctttgtggagggcaactcctggaccggcgcctggacctgggccgacgtcaatgacaacctgaaggctctgaccgacccccaggataagatcgtctacgagatgcaccagtatctcgactcggatggatccggcaccgcggagagctgcgtgtctaccacgattggtaaggagcgggtttcggccgcaacaaagtggctcaaggataacggcaaggttggcatcattggtgagttcgctggtggcgtcaatgatcagtgccggaccgctatttcaggaatgctggagtacttggctcagaacacagacgtgtggaagggagctctctggtgggcggctggcccctggtggggaaactatatgttcaacatggagcctccgagcggtgcagcttatgtgggcatgttggacatcttggagccctacctgggttga

SEQ ID NO: 20 - protein, 329 aa, Aspergillus fumigatus
MKFGSIVLIAAAAGFAVAAPAKRASVFQWFGSNESGAEFGENTIPGSYGKEFIFPDPSTISTLIGKGMNIFRIQFLMERLVPSSMTGSYNEEYLANLTSVVDAVTKAGSYAILDPHNFGRYNGQIISSTDDFKTFWQNLAGKFKSNNLVIFDTNNEYHDMDQTLVLNLNQAAINGIRAAGATSQYIFVEGNSWTGAWTWADVNDNLKALTDPQDKIVYEMHQYLDSDGSGTAESCVSTTIGKERVSAATKWLKDNGKVGIIGEFAGGVNDQCRTAISGMLEYLAQNTDVWKGALWWAAGPWWGNYMFNMEPPSGAAYVGMLDILEPYLG

SEQ ID NO: 21 - DNA, 1239 bp, Neosartorya fischeri
atgaaggcttcgactattatctgtgcacttctcccccttgctttggcggtgccgaatgcgaggcgggcttctgggtttgtttgtatgtttccctttcttctcttcatcgcagaagctgacggtgagatagggtttggaagtaacgagtctggcgccgagtttggagagaccaagctcccgggcgtgctggggacggattatatctggcccgatgcgtcgactatcaagactctgcatgatgccgggatgaacatcttccgtgttgcgttccggatggagaggctcatcccggataagatgacggggactccagatgcgacgtacatgaatgatctcaaggcgactgtcaatgcgattacgagtctgggggcgtatgcggtgattgatccccataactatggaagatactacgggaacatcatctcgtcgactgacgactttgctgcgttctggaagaccgtggctgcccagtttgcgtccaatgaccatgtcatttttgacaccaacaatgagtaccatgatatggaccagacgctcgttctcaacctcaaccaggctgccatcaacgccatccgtgctgcaggcgccacctcgcagtacatttttgtcgagggcaactcgtggtccggcgcgtggacctggaccaacgtcaacgacaacctcaaggccctcaccgaccctcaggataagatcgtctacgagatgcaccagtatctcgactcagacgggtccggcacgtcggccacctgcgtgagctccaccatcggccaggagcgcgtgcagtccgccacacagtggttgaagaccaatggtaagaaaggtatcataggcgagttcgctggaggccccaacagcgtgtgccagtccgctgtcacaggcatgcttgactacttgtctgccaactcggatgtgtggatgggcgcagcatggtgggccgctggtccctggtgggcagattatatgttcagcatggagccgccgtctggcactggctatcagaactatctctcgttgttgaagccgtatttcgtcggtggttcgggtggtaaccctccaacgaccaccacgacaactaccagcaagcctactacgaccactaccacggctgggaaccctggcggcaccggagtcgcacagcactggggccagtgtggtggaattggatggaagggtccgactgcctgcgccacgccatatacctgccagaagctgaacgactactactctcaatgcctgtag

SEQ ID NO: 22 - protein, 412 aa, Neosartorya fischeri
MKASTIICALLPLALAVPNARRASGFVCMFPFLLFIAEADGEIGFGSNESGAEFGETKLPGVLGTDYIWPDASTIKTLHDAGMNIFRVAFRMERLIPDKMTGTPDATYMNDLKATVNAITSLGAYAVIDPHNYGRYYGNIISSTDDFAAFWKTVAAQFASNDHVIFDTNNEYHDMDQTLVLNLNQAAINAIRAAGATSQYIFVEGNSWSGAWTWTNVNDNLKALTDPQDKIVYEMHQYLDSDGSGTSATCVSSTIGQERVQSATQWLKTNGKKGIIGEFAGGPNSVCQSAVTGMLDYLSANSDVWMGAAWWAAGPWWADYMFSMEPPSGTGYQNYLSLLKPYFVGGSGGNPPTTTTTTTSKPTTTTTTAGNPGGTGVAQHWGQCGGIGWKGPTACATPYTCQKLNDYYSQCL

SEQ ID NO: 23 - DNA, 999 bp, Aspergillus kawachii
atgaagtttcagagcactctgcttcttgccgccgcggctggttccgcgttggctgtgcctcatggtcctggacataagaagagggcgtctgtgtttgaatggttcggatcgaatgagtcgggtgctgagtttggaaccaatatccctggggtttggggaaccgactacatcttccccgacccctctgccatctctacgttgattgacaaggggatgaacttcttccgcgtccagttcatgatggagaggttgctgcccgactcgatgactggttcatatgatgaggagtatctggccaacttgacgacagtgataaaagcggtaacggatggaggcgcccatgcgcttgtcgaccctcataactatggcagatacaacggcgagatcatctccagcacgtcagacttccagaccttctgggagaacctggcgggccagtacaaagataacgacctggtcatgtttgacactaacaacgaatatcacgacatggaccaggatctcgtgctgaacctcaaccaagcagccattaacggcatccgcgccgcaggtgcgaccagccagtacatcttcgtcgaaggcaactcctggaccggcgcctggacgtgggtcgacgtcaacgacaacatgaagaatttgaccgaccccgaagacaagatcgtctatgaaatgcaccagtacctagactccgacggttccggcacttcggagacctgcgtgtccgagaccatcggaaaagagcgggtcactgaagctacacagtggctgaaggacaataagaaggtcggcttcataggcgaatatgccgggggttccaatgatgtatgtcggagtgccgtgtcggggatgctggagtacatggcgaataacaccgacgtatggaagggtgcgtcgtggtgggcagccgggccatggtggggagactacattttcagcatggagcccccagatggaactgcgtacacgggtatgctggatatcctggaggcatatctttga

SEQ ID NO: 24 - protein, 332 aa, Aspergillus kawachii
MKFQSTLLLAAAAGSALAVPHGPGHKKRASVFEWFGSNESGAEFGTNIPGVWGTDYIFPDPSAISTLIDKGMNFFRVQFMMERLLPDSMTGSYDEEYLANLTTVIKAVTDGGAHALVDPHNYGRYNGEIISSTSDFQTFWENLAGQYKDNDLVMFDTNNEYHDMDQDLVLNLNQAAINGIRAAGATSQYIFVEGNSWTGAWTWVDVNDNMKNLTDPEDKIVYEMHQYLDSDGSGTSETCVSETIGKERVTEATQWLKDNKKVGFIGEYAGGSNDVCRSAVSGMLEYMANNTDVWKGASWWAAGPWWGDYIFSMEPPDGTAYTGMLDILEAYL

SEQ ID NO: 25 - DNA, 1185 bp, Aspergillus clavatus
atgaagacgactactcttgttgctgcgcttctgcctctggtgctggcggcgccgaataccaagcgctcgtctgggtttgtctggttcggtgccaatgagtctggcgctgagtttggcgagagcaatctccctggcatcctcggcaaagactacatctggcccagtgcgtcgaccatccggacgctgcacgatggtggcatgaacgtcttccgtgtagccttccgcatggagcgattgatcccgaacaagatgactgggtctccggatggcacctacatgaatgatctgagatcgaccgttgaagcaatcaccagcttgggagcatatgctgtgattgacccccataactacggaagatactatggcaacatcatcacgtcgaccgacgactttgccgcgttctggaagaccctggcatccgagttcgcgtccaacgaccatgtcatcttcgacaccaacaacgagtacaacacgatggaccagaccctcgtcctcaacctcaaccaggccgccataaacgccatccgtgcttccggtgccacctcgcagtatatcttcgtcgagggcaactcgtggtccggcgcctggacctggaccgacatcaacgacaacatgaaggccctcaccgatccccaggataagatcgtgtacgagatgcaccagtacctcgactccgacggctccggcacctcgcccagctgcgtgagctcgaccatcggcaaggagcgcatggtggctgctacccagtggctcaaggataacaacaagaagggcttcctgggcgagtttgccggcgggcccaacgatgtctgcaagagcgccgtcacgggcatgctggactacatgtcgctcaacacggatgtctggatgggcgccgagtggtgggctgccggcccctggtgggcagactacatgttcagcatggagccgccatccggcactggctaccagaactatttcaatctgctgaagccttacttctctacctcctcctcctccccctccccctccaccaccaccactaccaaggcgactaccaccactaccactgccgcgggtcctaccggcaccggggtcgcccagccctgggctcagtgcggcggtagtggctacagcggtccgacacagtgcgcggcgccgtacacttgcacgaagcagaatgactggtactcgcagtgcctgtag

SEQ ID NO: 26 - protein, 394 aa, Aspergillus clavatus
MKTTTLVAALLPLVLAAPNTKRSSGFVWFGANESGAEFGESNLPGILGKDYIWPSASTIRTLHDGGMNVFRVAFRMERLIPNKMTGSPDGTYMNDLRSTVEAITSLGAYAVIDPHNYGRYYGNIITSTDDFAAFWKTLASEFASNDHVIFDTNNEYNTMDQTLVLNLNQAAINAIRASGATSQYIFVEGNSWSGAWTWTDINDNMKALTDPQDKIVYEMHQYLDSDGSGTSPSCVSSTIGKERMVAATQWLKDNNKKGFLGEFAGGPNDVCKSAVTGMLDYMSLNTDVWMGAEWWAAGPWWADYMFSMEPPSGTGYQNYFNLLKPYFSTSSSSPSPSTTTTTKATTTTTTAAGPTGTGVAQPWAQCGGSGYSGPTQCAAPYTCTKQNDWYSQCL

SEQ ID NO: 27 - DNA, 990 bp, Aspergillus fumigatus; identical to SEQ ID NO: 19

SEQ ID NO: 28 - protein, 329 aa, Aspergillus fumigatus; identical to SEQ ID NO: 20

SEQ ID NO: 29 - DNA, 1406 bp, Neosartorya fischeri
atgaggtttacgcagctcttcatcgcggtcagcacggcaagccttgctcttgctgcaccccctaagtccaaaaccaagagaacctcggccttccaatgtaagggacccatgtgtataaccagctcattcactgacttctgcagggtttggctgcaatgaatccggagcagagtttggaagtggcaacatccctggcgtcctgggcacagattacatctggcccaacacgaccgccattggcatcttgagggaagcagggatgaacatcttccgtgtgccatttctcatggagcgtctcgtccccaacaacatgactggaagccctgatgcgacgtacatggctgatctggtttcggtatgtccatgctcaagtgtatggccattgctgacgagtcagaccatcaactacatcacgtccaccggcgcgcacgccatcgtcgacccccacaactttggccgatagtaagtctaacctcccgcctggaatgaagacaccaactaagagaaccagctacggcaacatcatcacctcgaccagcgactttgccgccttctggacaaccgtcgccacgcaattcaaggacaacgacctcgtcatcttcgacaccagtaacactcacccccagcacccctcgttaaggctttaactaacccatccagacaatgaattcaacgccgaagaccaaaccaccgtcctagatctcaaccaagcagccatcaatgccatccgcgccgcaggcgccacatcgcagtacatcttcgtagaaggcaactcctggtccggcgcctggacctgggattctgttaatgacaacctgaagacacttaccgaccccgtcgccgattccaacaaactcatctacgaaatgcaccagtacctcgacagtgatggctccggcacctcggagtcatgcgtctccgcgacaatcgggcaagagcgtctcgcctccgcgacggcttggctgattgctaacgccaaaaaagcgtttctgggtgagttcgctgcaggcgcgaatagtgtttgtgcgagtgcggtgaccgggatgttggagtatatggaggcgaatgcgaatgtttggcttggggcggagtggtgggctgctgggccgtggtggggggattatatctatagtatggagccgacggccgggatggcgtatgagtattatctggaggttttggagccgtattttcctggggggagttatacggcgacgtcgacggggacggggactgctacggcgactgctacgagtacgagctcgacgacagcggtaacagggacgtccactgcagttgcgcagcattggggtcaatgtggtggtcagggctggacggggccgacgggctgtgagtccccgtatacttgccaggtgcagaacgcctattattcgcagtgtttgtag

SEQ ID NO: 30 - protein, 405 aa, Neosartorya fischeri
MRFTQLFIAVSTASLALAAPPKSKTKRTSAFQWFGCNESGAEFGSGNIPGVLGTDYIWPNTTAIGILREAGMNIFRVPFLMERLVPNNMTGSPDATYMADLVSTINYITSTGAHAIVDPHNFGRYYGNIITSTSDFAAFWTTVATQFKDNDLVIFDTNNEFNAEDQTTVLDLNQAAINAIRAAGATSQYIFVEGNSWSGAWTWDSVNDNLKTLTDPVADSNKLIYEMHQYLDSDGSGTSESCVSATIGQERLASATAWLIANAKKAFLGEFAAGANSVCASAVTGMLEYMEANANVWLGAEWWAAGPWWGDYIYSMEPTAGMAYEYYLEVLEPYFPGGSYTATSTGTGTATATATSTSSTTAVTGTSTAVAQHWGQCGGQGWTGPTGCESPYTCQVQNAYYSQCL

SEQ ID NO: 31 - DNA, 1002 bp, Metazoa
atgaaattccgtaaccttttctttgctgccgtagctggctctgcggttgctgctcctctggctaaggagcaaaaaaagagggattcagttttccaatggatcggcgccaatgagtcgggtgctgaattcggcgagaacaacctccctggcgtatggggtacagattatatcttcccggatgtctcggccattaccaccttgattgataaggggatgaatatattccgtatccaattcaagatggagagattggttccagactcaatgacgggggcatacgatgaggcctacctgcagaacttgaccacggtggtcaatgccgtcactgatgcgggtgtccatgctatcttggacccccataactatggcagattcaatggcgagatcatgtccactccatctgatttccaaacattctggaagaacttggcagaacagttccagagcaactctttggttatcttcgataccaacaatgaataccatgacatggaccaagagctggtcctgaacctcaaccaagccgccattgacgggatccgagaggctggcgccacggagcaatatatcttcgttgaaggtaactcgtacaccggggcctggacctggaccgatgtgaacgacaacatgaagaacttggaggatccccaggataagatcgtctatcagatgcaccagtatttggactcggatgggtctggtacttccgagacctgcgtgtctggtaccattggccaggagcgtgtcaccagcgctactcaatggctcaaggacaacaagaaggtcggtatcatcggtgagttcgcgggtggaaacaacgaccagtgcaaaactgccgtgaagggaatgctggattatctggctgagaacaccgacgtgtggaagggtgcgctttggtgggcggccggtccatggtggggagattatatgtacagtttggagcctccgaacggagttgcgttcacgggaatgttggacgttctgcaggcatacctggggtag

SEQ ID NO: 32 - protein, 410 aa, Metazoa
MKTLSLVAVLLVQAWTASSSPRAKRSSGFQWFGSNESGAEFGSGNIPGTLGTDYIWPDATAIETLHSAGMNIFRVPFLMERLVPNELTGAADATYMADLKATVQTITDLGAYAVVDPHNFGRYYGSIITSTSDFAAFWTTVASEFTDNDRVIFDTNNEFHDEDQTLVLNLNQAAITAIRATGATTQYIFVEGNSYTGAWTWTTTNTNLVNLTDPNDHLIYEMHQYLDSDGSGTSSTCVSSTIGAERIADATSWLRENGKQAVLGEFAGGANSVCEEAVTGMLDAMVDASDVWLGGMWWSAGPWWGDYIFSLEPDDGIAYEYYLSVLEGYTPGSSSSGSSTTSAVTSASSTSAAGSATTSKVTTTTAAATATSTAVAAHYAQCGGQGWTGATTCADGYTCTVENAWYSQCL

SEQ ID NO: 33 - DNA, 999 bp, Aspergillus aculeatus
atgaagctgcttaacctacttgttgctgctgccgcggccggcagtgcagtggcggccccgacccacgaacacacgaagcgggcttctgttttcgaatggatcggatccaatgagtcggacgccgagttcggcaccgccattccgggaacttggggtatcgactacatcttcccggatacctccgccatcgcgactctcgtcagcaagggcatgaacatcttccgcgtgcagttcatgatggaaagattggttccgaactcgatgacggggtcgtatgacgatgcctacctgaacaatctcacgacggtggtgaatgcaatcgcggctgcaggtgtccatgccattgtagaccctcataactatggacgatacaacaacgagatcatctccagcaccgccgacttccagaccttctggcagaacctcgccgggcagttcaaggacaacgacctggtcatcttcgacacgaacaacgaatacaacaccatggaccaaaccctggtgctggacctcaaccaggcggcgatcgacggcatccgggccgccggcgccacttcccagtacatcttcgcggagggcaactcgtggtccggggcgtggacctgggcggacatcaacgacaatatgaaggcgctgaccgacccgcaggacaagctcgtctacgagatgcaccagtacctggactcggacggctcagggacgtccggcgtctgcgtgtcggagacgatcggcgcggaacgcctgcaggccgccacacagtggctgaaggacaacggcaaggtggacatcctgggcgagtacgccggcggggccaacgatgtctgtcgcacggccatcgcggggatgctggagtatatggccaacaacacggatgtgtggaagggcgcggtttggtggacggctggcccctggtgggcggattacatgttcagcatggagccgcccagcgggccggcttatagcgggatgctggacgttctggagccgtatctggggtga

SEQ ID NO: 34 - protein, 332 aa, Aspergillus aculeatus
MKLLNLLVAAAAAGSAVAAPTHEHTKRASVFEWIGSNESDAEFGTAIPGTWGIDYIFPDTSAIATLVSKGMNIFRVQFMMERLVPNSMTGSYDDAYLNNLTTVVNAIAAAGVHAIVDPHNYGRYNNEIISSTADFQTFWQNLAGQFKDNDLVIFDTNNEYNTMDQTLVLDLNQAAIDGIRAAGATSQYIFAEGNSWSGAWTWADINDNMKALTDPQDKLVYEMHQYLDSDGSGTSGVCVSETIGAERLQAATQWLKDNGKVDILGEYAGGANDVCRTAIAGMLEYMANNTDVWKGAVWWTAGPWWADYMFSMEPPSGPAYSGMLDVLEPYLG

SEQ ID NO: 35 - DNA, 1002 bp, Aspergillus oryzae; identical to SEQ ID NO: 31

SEQ ID NO: 36 - protein, 333 aa, Aspergillus oryzae
MKFRNLFFAAVAGSAVAAPLAKEQKKRDSVFQWIGANESGAEFGENNLPGVWGTDYIFPDVSAITTLIDKGMNIFRIQFKMERLVPDSMTGAYDEAYLQNLTTVVNAVTDAGVHAILDPHNYGRFNGEIMSTPSDFQTFWKNLAEQFQSNSLVIFDTNNEYHDMDQELVLNLNQAAIDGIREAGATEQYIFVEGNSYTGAWTWTDVNDNMKNLEDPQDKIVYQMHQYLDSDGSGTSETCVSGTIGQERVTSATQWLKDNKKVGIIGEFAGGNNDQCKTAVKGMLDYLAENTDVWKGALWWAAGPWWGDYMYSLEPPNGVAFTGMLDVLQAYLG

SEQ ID NO: 37 - DNA, 1228 bp, Emericella nidulans
atgaggtctctcgtccttctgtcgtccgtcctggccctggtggcaccctccaaaggcgccttcacatgtatatctcacacccctcgcttctccagatatctctacctgtgctaaaatcatgcctgtaaatcagggcttggtaccaacgaagccggtgccgaattcggcgagggctcctaccccggcgaactgggcacggaatacatttggcctgacctgggtacgattggtacgctgaggaacgaggggatgaacatattccgcgttgcgttctccatggagcgcctggtgcctgattcgttggctggaccggtagcggacgagtattttcaggacttggtcgaggtgcgcctttcttctctcttgcctcagatcttgggcatggcccaagaacagcaagtcgctaactgcatcagacggtcaacggcattacggccctgggtgcgtatgcagtccttgacccgcataattatggacggtagtatgtatccctagtcaacagaccctacgaacgggccagcaatgctgacgagctaaagctacggcaacattatcacttcaaccgatgactttgcagccttctggaccatccttgccaccgagttcgcttctaatgagcttgtcatcttcgataccagtccgtttattccgacatccactcacttgtgttcttgactgacaaaaatcagataatgaataccacaccatggaccagtcccttgtcctgaacctcaatcaggcggccatagacgctattcgcgcctccggtgccaccagccaatatatcttcgcggaaggaaactcttggactggagcctggacctgggtggatgtcaacgacaacatgaaagcgctcaccgatccccaagataagctcatctacgagatgcaccagtatcttgactccgacggctctgggacgaacacggcctgcgtcagttctactatcggcagcgaacgcgtgacagccgctacaaactggctgcgagagaacgggaaactgggggtgctcggtgagtttgcaggcgcaaacaaccaggtctgcaaggacgcagtcgcagatctgctggagtatcttgaggaaaacagtgatgtatggcttggggccctttggtgggctgctgggccttggtggggtgattatatgttcaacatggagcctacttcgggtattgcataccaggagtactcggaaatcttgcagccgtatttcgtgggaagtcaataa

SEQ ID NO: 38 - protein, 326 aa, Emericella nidulans
MRSLVLLSSVLALVAPSKGAFTWLGTNEAGAEFGEGSYPGELGTEYIWPDLGTIGTLRNEGMNIFRVAFSMERLVPDSLAGPVADEYFQDLVETVNGITALGAYAVLDPHNYGRYYGNIITSTDDFAAFWTILATEFASNELVIFDTNNEYHTMDQSLVLNLNQAAIDAIRASGATSQYIFAEGNSWTGAWTWVDVNDNMKALTDPQDKLIYEMHQYLDSDGSGTNTACVSSTIGSERVTAATNWLRENGKLGVLGEFAGANNQVCKDAVADLLEYLEENSDVWLGALWWAAGPWWGDYMFNMEPTSGIAYQEYSEILQPYFVGSQ

SEQ ID NO: 39 - DNA, 1170 bp, Myceliophthora thermophila
atgaagtcctccatcctcgccagcgtcttcgccacgggcgccgtggctcaaagtggtccgtggcagcaatgtggtggcatcggatggcaaggatcgaccgactgtgtgtcgggctaccactgcgtctaccagaacgattggtacagccagtgcgtgcctggcgcggcgtcgacaacgctgcagacatcgaccacgtccaggcccaccgccaccagcaccgcccctccgtcgtccaccacctcgcctagcaagggcaagctgaagtggctcggcagcaacgagtcgggcgccgagttcggggagggcaattaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgaccagggttacctccgcaacctgaccgaggtggtcaacttcgtgacgaacgcgggcaagtacgccgtcctggacccgcacaactacggccggtactacggcaacatcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaccatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcttgaagaagtacttgccgtaa

SEQ ID NO: 40 - protein, 389 aa, Myceliophthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDQGYLRNLTEVVNFVTNAGKYAVLDPHNYGRYYGNIITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSTIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 41 - DNA, 20 bp, Artificial Sequence
  Artificial DNA Primer
  misc_feature (3)..(3): N= T OR C
  misc_feature (6)..(6): N= A OR G
  misc_feature (9)..(9): N= T, C, A OR G
  misc_feature (12)..(12): N= T, C, A OR G
  misc_feature (15)..(15): N= T, C, A OR G
aangantcnggngcngagtt

SEQ ID NO: 42 - DNA, 20 bp, Artificial Sequence
  Artificial DNA Primer
  misc_feature (3)..(3): N= T OR C
  misc_feature (6)..(6): N= A OR G
  misc_feature (9)..(9): N= T OR C
  misc_feature (12)..(12): N= T, C, A OR G
  misc_feature (15)..(15): N= T, C, A OR G
aanganagnggngcngaatt

SEQ ID NO: 43 - DNA, 18 bp, Artificial Sequence
  Artificial DNA Primer
aagatgtactgggaagtg

SEQ ID NO: 44 - DNA, 21 bp, Artificial Sequence
  Artificial DNA Primer
tggttgagattgaggactaag

SEQ ID NO: 45 - DNA, 19 bp, Artificial Sequence
  Artificial DNA Primer
agagccggtcattgagttg

SEQ ID NO: 46 - DNA, 20 bp, Artificial Sequence
  Artificial DNA Primer
atgaagctcggctctctcgt

SEQ ID NO: 47 - DNA, 90 bp, Artificial Sequence
  Artificial DNA Primer
ttcacgcccaaagcataaacgacgtaatacgactcactatagggagaccacaagaaggagatatacatatggcgaaagtattccaatggt

SEQ ID NO: 48 - DNA, 52 bp, Artificial Sequence
  Artificial DNA Primer
gggctagttattgctcagcggttattaaagatacggagtcaaaataggaagt

SEQ ID NO: 49 - DNA, 24 bp, Artificial Sequence
  Artificial DNA Primer
ttcacgcccaaagcataaacgacg

SEQ ID NO: 50 - DNA, 22 bp, Artificial Sequence
  Artificial DNA Primer
ggggctagttattgctcagcgg

SEQ ID NO: 51 - DNA, 42 bp, Artificial Sequence
  Artificial DNA Primer
tacttccaatccaatgcagcgaaagtattccaatggttcggt

SEQ ID NO: 52 - DNA, 46 bp, Artificial Sequence
  Artificial DNA Primer
ttatccacttccaatgttattaaagatacggagtcaaaataggaag

SEQ ID NO: 53 - DNA, 48 bp, Artificial Sequence
  Artificial DNA Primer
aatcctctatatacacaactggatttacatgaagctcggctctctcgt

SEQ ID NO: 54 - DNA, 42 bp, Artificial Sequence
  Artificial DNA Primer
gaaagcaggagaccaagcgtgcgaaagtattccaatggttcg

SEQ ID NO: 55 - DNA, 41 bp, Artificial Sequence
  Artificial DNA Primer
cgaaccattggaatactttcgcacgcttggtctcctgcttt

SEQ ID NO: 56 - DNA, 55 bp, Artificial Sequence
  Artificial DNA Primer
caggtgtcagtcacctctagttaattaatcaaagatacggagtcaaaataggaag